SEQUENCE LISTING (40 sequences)

SEQ ID NO: 1 - DNA, 429 bp, Artificial Sequence
  Synthetic primer
atggaaaggcactggatctttctcttcctgatttcagtaactgcaggtgtccactcccaggtccaacttcagcagtctggggctgaactggcaaaacctggggcctcagtgaagatgtcctgcaaggcgtctggctacacctttactagctactggatgcactgggtaaaacagaggcctggacagggtctggaatggattggatacattaatcctagcactggttatattgagtacaatcagaacttcaaggacaaggccacattgactgcagacaaatcctccagcacagcctacatgcaactgagcagcctgacatctgaggactctgcagtctattactgtgcaagatccccctctcattattacggtagtaggtacggatatttcgatgtctggggcgcagggaccacggtcaccgtctcctca

SEQ ID NO: 2 - protein, 143 aa, Artificial Sequence
  Synthetic peptide
MERHWIFLFLISVTAGVHSQVQLQQSGAELAKPGASVKMSCKASGYTFTSYWMHWVKQRPGQGLEWIGYINPSTGYIEYNQNFKDKATLTADKSSSTAYMQLSSLTSEDSAVYYCARSPSHYYGSRYGYFDVWGAGTTVTVSS

SEQ ID NO: 3 - DNA, 384 bp, Artificial Sequence
  Synthetic primer
atggattttcaagtgcagattttcagcttcctgctaatcagtgcttcagtcataatgtccagaggacaaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgtcagcagtggagtagtaacccacggacgttcggtggaggcaccaagctggaaatcaaa

SEQ ID NO: 4 - protein, 128 aa, Artificial Sequence
  Synthetic peptide
MDFQVQIFSFLLISASVIMSRGQIVLSQSPAILSASPGEKVTMTCRASSSVSYMHWYQQKPGSSPKPWIYATSNLASGVPARFSGSGSGTSYSLTISRVEAEDAATYYCQQWSSNPRTFGGGTKLEIK

SEQ ID NO: 5 - DNA, 357 bp, Artificial Sequence
  Synthetic primer
gaggttcagctgcagcagtctggggcagaacttgtgaagccaggggcctcagtcaagttgtcctgcacaacttctggcttcaacattatagacacctatatacactgggtgaatcagaagcctgatcagggcctggagtggattggaaggattgaccctgcggatggtaatagaaaatctgacccgaagttccaggtcaaggccacaataactgttgacacatcctccaacacagcctacctgcaactcagcagcctgacatctggggacactgccgtctattactgtgccagagggtacggtaacctcaatgctatggactcctggggtcaaggaacctcagtcaccgtctcctca

SEQ ID NO: 6 - protein, 138 aa, Artificial Sequence
  Synthetic peptide
MKCSWVIFFLMAVVTGVNSEVQLQQSGAELVKPGASVKLSCTTSGFNIIDTYIHWVNQKPDQGLEWIGRIDPADGNRKSDPKFQVKATITVDTSSNTAYLQLSSLTSGDTAVYYCARGYGNLNAMDSWGQGTSVTVSS

SEQ ID NO: 7 - DNA, 381 bp, Artificial Sequence
  Synthetic primer
atgagggtccttgctgagctcctggggctgctgctgttctgctttttaggtgtgagatgtgacatccagatgaaccagtctccatccagtctgtctgcatcccttggggacacaattaccatcacttgccatgccagtcagaacattaatgtttggttaaactggtatcagcagaaacccggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcaaagttatcctctgacgttcggtggaggcaccaagctggaaatcaaa

SEQ ID NO: 8 - protein, 127 aa, Artificial Sequence
  Synthetic peptide
MRVLAELLGLLLFCFLGVRCDIQMNQSPSSLSASLGDTITITCHASQNINVWLNWYQQKPGNIPKLLIYKASNLHTGVPSRFSGSGSGTGFTLTISSLQPEDIATYYCQQGQSYPLTFGGGTKLEIK

SEQ ID NO: 9 - protein, 10 aa, Artificial Sequence
  Synthetic peptide
GYTFTSYWMH

SEQ ID NO: 10 - protein, 17 aa, Artificial Sequence
  Synthetic peptide
YINPSTGYIEYNQNFKD

SEQ ID NO: 11 - protein, 15 aa, Artificial Sequence
  Synthetic peptide
SPSHYYGSRYGYFDV

SEQ ID NO: 12 - protein, 10 aa, Artificial Sequence
  Synthetic peptide
RASSSVSYMH

SEQ ID NO: 13 - protein, 7 aa, Artificial Sequence
  Synthetic peptide
ATSNLAS

SEQ ID NO: 14 - protein, 9 aa, Artificial Sequence
  Synthetic peptide
QQWSSNPRT

SEQ ID NO: 15 - protein, 10 aa, Artificial Sequence
  Synthetic peptide
GFNIIDTYIH

SEQ ID NO: 16 - protein, 17 aa, Artificial Sequence
  Synthetic peptide
RIDPADGNRKSDPKFQV

SEQ ID NO: 17 - protein, 10 aa, Artificial Sequence
  Synthetic peptide
GYGNLNAMDS

SEQ ID NO: 18 - protein, 11 aa, Artificial Sequence
  Synthetic peptide
HASQNINVWLN

SEQ ID NO: 19 - protein, 7 aa, Artificial Sequence
  Synthetic peptide
KASNLHT

SEQ ID NO: 20 - protein, 9 aa, Artificial Sequence
  Synthetic peptide
QQGQSYPLT

SEQ ID NO: 21 - DNA, 1416 bp, Artificial Sequence
  Synthetic primer
gccgccaccatgggctggagctggatcctgctgttcctcctgagcgtgacagcaggagtgcacagccaggtgcagctggtgcagtctggggctgaggtgaagaagcctggggctacagtgaaaatctcctgcaaggtttctggatacaccttcaccgacacctatatacactgggtgcaacaggcccctggaaaagggcttgagtggatgggaaggattgaccctgcggatggtaatagaaaatctgacccgaagttccaggtcagagtcaccataaccgcggacacgtctacagacacagcctacatggagctgagcagcctgagatctgaggacacggccgtgtattactgtgcaagagggtacggtaacctcaatgctatggactcctggggccaaggtaccctggtcaccgtgtcgagagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 22 - protein, 471 aa, Artificial Sequence
  Synthetic peptide
AATMGWSWILLFLLSVTAGVHSQVQLVQSGAEVKKPGATVKISCKVSGYTFTDTYIHWVQQAPGKGLEWMGRIDPADGNRKSDPKFQVRVTITADTSTDTAYMELSSLRSEDTAVYYCARGYGNLNAMDSWGQGTLVTVSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1416 bp, Artificial Sequence
  Synthetic primer
gccgccaccatgggctggagctggatcctgctgttcctcctgagcgtgacagcaggagtgcacagccaggtgcagctggtgcagtctggggctgaggtgaagaagcctggggcctcagtgaaggtttcctgcaaggcatctggatacaccttcaccgacacctatatacactgggtgcgacaggcccctggacaagggcttgagtggatgggaaggattgaccctgcggatggtaatagaaaatctgacccgaagttccaggtcagagtcaccatgaccagggacacgtccacgagcacagtctacatggagctgagcagcctgagatctgaggacacggccgtgtattactgtgcgagagggtacggtaacctcaatgctatggactcctggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 24 - protein, 471 aa, Artificial Sequence
  Synthetic peptide
AATMGWSWILLFLLSVTAGVHSQVQLVQSGAEVKKPGASVKVSCKASGYTFTDTYIHWVRQAPGQGLEWMGRIDPADGNRKSDPKFQVRVTMTRDTSTSTVYMELSSLRSEDTAVYYCARGYGNLNAMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 1416 bp, Artificial Sequence
  Synthetic primer
gccgccaccatgggctggagctggatcctgctgttcctcctgagcgtgacagcaggagtgcacagccaggtccagcttgtgcagtctggggctgaggtgaagaagcctggggcctcagtgaaggtttcctgcaaggcttctggatacaccttcactgacacctatatacactgggtgcgccaggcccccggacaaaggcttgagtggatgggaaggattgaccctgcggatggtaatagaaaatctgacccgaagttccaggtcagagtcaccattaccagggacacatccgcgagcacagcctacatggagctgagcagcctgagatctgaagacacggctgtgtattactgtgcgagagggtacggtaacctcaatgctatggactcctggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - protein, 471 aa, Artificial Sequence
  Synthetic peptide
AATMGWSWILLFLLSVTAGVHSQVQLVQSGAEVKKPGASVKVSCKASGYTFTDTYIHWVRQAPGQRLEWMGRIDPADGNRKSDPKFQVRVTITRDTSASTAYMELSSLRSEDTAVYYCARGYGNLNAMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1416 bp, Artificial Sequence
  Synthetic primer
gccgccaccatgggctggagctggatcctgctgttcctcctgagcgtgacagcaggagtgcacagccaggtccagctggtacagtctggggctgaggtgaagaagcctggggcctcagtgaaggtctcctgcaaggtttccggatacaccctcactgacacctatatacactgggtgcgacaggctcctggaaaagggcttgagtggatgggaaggattgaccctgcggatggtaatagaaaatctgacccgaagttccaggtcagagtcaccatgaccgaggacacatctacagacacagcctacatggagctgagcagcctgagatctgaggacacggccgtgtattactgtgcaacagggtacggtaacctcaatgctatggactcctggggccaagggacaatggtcaccgtctcttcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 28 - protein, 471 aa, Artificial Sequence
  Synthetic peptide
AATMGWSWILLFLLSVTAGVHSQVQLVQSGAEVKKPGASVKVSCKVSGYTLTDTYIHWVRQAPGKGLEWMGRIDPADGNRKSDPKFQVRVTMTEDTSTDTAYMELSSLRSEDTAVYYCATGYGNLNAMDSWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1416 bp, Artificial Sequence
  Synthetic primer
gccgccaccatgggctggagctggatcctgctgttcctcctgagcgtgacagcaggagtgcacagccaggtgcagctgcaggagtcgggcccaggactggtgaagccttcggagaccctgtccctcacctgcactgtctctggtggctccatcagtgacacctatatacactggatccggcagcccccagggaagggactggagtggattgggaggattgaccctgcggatggtaatagaaaatctgacccgaagttccaggtccgagtcaccatatcagtagacacgtccaagaaccagttctccctgaagctgagctctgtgaccgctgcggacacggccgtgtattactgtgcgagagggtacggtaacctcaatgctatggactcctggggccaagggacaatggtcaccgtctcttcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 30 - protein, 470 aa, Artificial Sequence
  Synthetic peptide
AATMGWSWILLFLLSVTAGVHSQVQLQESGPGLVKPSETLSLTCTVSGGSISDTYIHWIRQPPGKGLEWIGRIDPADGNRKSDPKFQVRVTISVDTSKNQFSLKLSSVTAADTAVYYCARGYGNLNAMDSWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 711 bp, Artificial Sequence
  Synthetic primer
gccgccaccatgggctggagctggatcctgctgttcctcctgagcgtgacagcaggagtgcacagcgacatccagatgacccagtctccatcctccctgtctgcatctgtaggagacagagtcaccatcacttgccatgccagtcagaacattaatgtttggttaaactggtatcagcagaaaccagggaaagcccctaagctcctgatctacaaggcttccaacttgcacacaggggtcccatcaaggttcagtggaagtggatctgggacagattttactttcaccatcagcagcctgcagcctgaagatattgcaacatattactgtcaacagggtcaaagttatcctctgacgttcggcggagggaccaagctggagatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 32 - protein, 236 aa, Artificial Sequence
  Synthetic peptide
AATMGWSWILLFLLSVTAGVHSDIQMTQSPSSLSASVGDRVTITCHASQNINVWLNWYQQKPGKAPKLLIYKASNLHTGVPSRFSGSGSGTDFTFTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - DNA, 711 bp, Artificial Sequence
  Synthetic primer
gccgccaccatgggctggagctggatcctgctgttcctcctgagcgtgacagcaggagtgcacagcgacatccagatgacccagtctccatcctccctgtctgcatctgtaggagacagagtcaccatcacttgccatgccagtcagaacattaatgtttggttaaactggtatcagcagaaaccagggaaagcccctaagctcctgatctataaggcttccaacttgcacacaggggtcccatcaaggttcagtggcagtggatctgggacagatttcactctcaccatcagcagtctgcaacctgaagattttgcaacttactactgtcaacagggtcaaagttatcctctgacgttcggcggagggaccaagctggagatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 34 - protein, 236 aa, Artificial Sequence
  Synthetic peptide
AATMGWSWILLFLLSVTAGVHSDIQMTQSPSSLSASVGDRVTITCHASQNINVWLNWYQQKPGKAPKLLIYKASNLHTGVPSRFSGSGSGTDFTLTISSLQPEDFATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 711 bp, Artificial Sequence
  Synthetic primer
gccgccaccatgggctggagctggatcctgctgttcctcctgagcgtgacagcaggagtgcacagcgacatccagatgacccagtctccatcctccctgtctgcatctgtaggagacagagtcaccatcacttgccatgccagtcagaacattaatgtttggttaaactggtatcagcagaaaccagggaaagttcctaagctcctgatctataaggcttccaacttgcacacaggggtcccatctcggttcagtggcagtggatctgggacagatttcactctcaccatcagcagcctgcagcctgaagatgttgcaacttattactgtcaacagggtcaaagttatcctctgacgttcggcggagggaccaaggtggagatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 36 - protein, 236 aa, Artificial Sequence
  Synthetic peptide
AATMGWSWILLFLLSVTAGVHSDIQMTQSPSSLSASVGDRVTITCHASQNINVWLNWYQQKPGKVPKLLIYKASNLHTGVPSRFSGSGSGTDFTLTISSLQPEDVATYYCQQGQSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 711 bp, Artificial Sequence
  Synthetic primer
gccgccaccatgggctggagctggatcctgctgttcctcctgagcgtgacagcaggagtgcacagcgacatccaggtgacccagtctccatccttcctgtctgcatctgtaggagacagagtcaccatcacttgccatgccagtcagaacattaatgtttggttaaactggtatcagcaaaaaccagggaaagcccctaagctcctgatctataaggcttccaacttgcacacaggggtcccatcaaggttcagcggcagtggatctgggacagaattcactctcacaatcagcagcctgcagcctgaagattttgcaacttattactgtcaacagggtcaaagttatcctctgacgttcggcggagggaccaaggtggagatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 38 - protein, 236 aa, Artificial Sequence
  Synthetic peptide
AATMGWSWILLFLLSVTAGVHSDIQVTQSPSFLSASVGDRVTITCHASQNINVWLNWYQQKPGKAPKLLIYKASNLHTGVPSRFSGSGSGTEFTLTISSLQPEDFATYYCQQGQSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 711 bp, Artificial Sequence
  Synthetic primer
gccgccaccatgggctggagctggatcctgctgttcctcctgagcgtgacagcaggagtgcacagcgacatccagatgacccagtctccatcctccctgtctgcatctgtaggagacagagtcaccatcacttgccatgccagtcagaacattaatgtttggttaaactggtatcagcagaaaccagggaaagcccctaagcgcctgatctataaggcttccaacttgcacacaggggtcccatcaaggttcagcggcagtggatctgggacagaattcactctcacaatcagcagcctgcagcctgaagattttgcaacttattactgtcaacagggtcaaagttatcctctgacgttcggcggagggaccaaggtggagatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 40 - protein, 236 aa, Artificial Sequence
  Synthetic peptide
AATMGWSWILLFLLSVTAGVHSDIQMTQSPSSLSASVGDRVTITCHASQNINVWLNWYQQKPGKAPKRLIYKASNLHTGVPSRFSGSGSGTEFTLTISSLQPEDFATYYCQQGQSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2016/0311918 A1
(43) Pub. Date: Oct. 27, 2016

(54) Anti-Ron Monoclonal Antibodies as a Cytotoxic Drug Delivery System for Targeted Cancer Therapy

(71) Applicant: TEXAS TECH UNIVERSITY SYSTEM, Lubbock, TX (US)

(72) Inventors: Ming-Hai Wang, Amarillo, TX (US); Hang-Ping Yao, Zhejiang (CN)

(21) Appl. No.: 15/105,445

(22) PCT Filed: Dec. 15, 2014
(86) PCT No.: PCT/US2014/070248

§ 371 Date: Jun. 16, 2016

Related U.S. Application Data
(60) Provisional application No. 62/057,367, filed on Sep. 30, 2014.
(60) Provisional application No. 61/916,519, filed on Dec. 16, 2013.

(51) Int. Cl. C07K 16/30; A61K 31/282; A61K 31/513; A61K 31/7068; A61K 47/48; A61K 39/395

(52) U.S. Cl. CPC C07K 16/30; A61K 47/48384; A61K 47/48569; A61K 39/39558; A61K 31/513; A61K 31/7068; A61K 31/282; C07K 2317/24

(57) ABSTRACT

The present invention includes unique, isolated monoclonal antibodies that bind human RON, and methods for making and using the same.

### TECHNICAL FIELD OF THE INVENTION

The present invention relates in general to the field of monoclonal antibodies, and more particularly, to anti-RON monoclonal antibodies as a cytotoxic drug delivery system for targeted cancer therapy.

### BACKGROUND OF THE INVENTION

Without limiting the scope of the invention, its background is described in connection with RON (recepteur d'origine nantais).

Since their discovery in the early 1990s, the pathogenic roles of RON in cancer biology have been extensively studied under various genetic, biochemical, and biological models. Preclinical evidence from both in vitro and in vivo experiments has revealed that RON signaling is integrated at variable levels into the cellular growth and invasive machinery in different types of epithelial cancers. Moreover, aberrant RON expression, characterized by protein overexpression and generation of oncogenic variants, is featured specifically in cancers derived from colon, breast, and pancreatic tissues. Aberrant RON activation regulates invasive cellular growth and facilitates malignant tumor progression. In light of these findings, targeting RON signaling by small molecules and therapeutic antibodies is under intensive investigation, laying the foundation for future clinical validation. Currently, various preclinical experiments have been evaluated. Clinical trials using small molecule inhibitors and therapeutic antibodies are also conducted.

The RON receptor tyrosine kinase is a potential drug target. Various types of tumors including breast and pancreatic cancers displayed aberrant RON expression featured by overexpression, isoform generation, and constitutive activation. Specific antibodies bind to RON on the surface of cancerous cells and cause RON internalization. This process is effective to deliver cytotoxic drugs for cancer treatment.

The present inventors have also published on the role of RON in oncogenesis, namely, Wang, et al., “Oncogenesis of RON receptor tyrosine kinase: a molecular target for malignant epithelial cancers”, Acta Pharmacologica Sinica (2006) 27, 641-650, which is the first publication noting and demonstrating the target potential of the RON receptor using monoclonal antibodies to inhibit RON and oncogenesis. Wang, et al., also published a manuscript on the role of RON, entitled, “RON Receptor Tyrosine Kinase as a Target for Delivery of Chemodrugs by Antibody Directed Pathway for Cancer Cell Cytotoxicity”, Mol. Pharmaceutics, 2010, 7 (2), pp. 386-397, in which unique anti-RON antibodies were used in conjunction with previously described drug-loaded PEG containing liposomes, which demonstrated in vivo antitumorigenic effects.

Another RON targeting molecule is taught in U.S. Pat. No. 8,133,489, issued to Pereira, et al., entitled “Inhibition of macrophage-stimulating protein receptor (RON) and methods of treatment thereof” Briefly, the disclosure is directed to antibodies or fragments thereof, including human antibodies, specific for Macrophage-Stimulating Protein Receptor (MSP-R or RON), which inhibited RON activation. Also provided are methods to inhibit RON, particularly the use of RON antibodies to treat diseases such as cancer.

Pereira, D. S., et al, also published “Therapeutic implications of a human neutralizing antibody to the macrophage-stimulating protein receptor tyrosine kinase (RON), a c-MET family member”, Cancer Research, Volume 66, Issue 18, 15 Sep. 2006, Pages 9162-9170. This publication discusses anti-RON antibodies in vivo efficacy against tumor xenographs, in which anti-RON antibodies were made through phage display.

Pereira, et al., also filed United States Patent Application Publication No. 20090246205, entitled, “Inhibition of macrophage-stimulating protein receptor (ron)”, which was directed to methods for treatment of tumors and other diseases in a mammal comprising administration of antibodies specific for Macrophage-Stimulating Protein Receptor (“MSP-R” or “RON”). Compositions comprising antibodies or antibody fragments specific for RON, including human antibodies, that inhibit RON activation are also said to be disclosed.

Whalen, et al., filed United States Patent Application Publication No. 20120027773, entitled Anti-RON antibodies, which is said to teach monoclonal antibodies that bind and inhibit activation of human RON (Recepteur d' Origine Nantais). The antibodies area said to be useful for treating certain forms of cancer that are associated with activation of RON.

Huet, et al., filed United States Patent Application Publication No. 20090226442, entitled, “RON antibodies and uses thereof”. Briefly, this application is said to teach antibodies that bind to RON (MST1R), and uses thereof. In particular in the diagnosis and treatment of cancer, the antibodies inhibit RON-mediated pro-survival and tumor proliferation pathways, and variants, fragments, and derivatives thereof. Also taught are antibodies that block the ability of the ligand, MSP to bind to RON, as well as fragments, variants and derivatives of such antibodies. The invention also includes polynucleotides encoding the above antibodies or fragments, variants or derivatives thereof, as well as vectors and host cells comprising such polynucleotides.

The invention further includes methods of diagnosing and treating cancer using the antibodies of the invention.

Although antibodies that bind RON are known in the art, there is still a need for improved RON antibodies that can be used as therapeutic agents.

### SUMMARY OF THE INVENTION

In one embodiment, the present invention includes an isolated monoclonal antibody that binds human RON, comprising a monoclonal antibody selected from Zt/g4-DM1, Zt/c1-DM1, Zt/64, 3F12, B9, 1G4, or Zt/f2. In one aspect, the monoclonal antibody comprises complementarity determining region (CDR) sequences interposed between human and humanized framework sequences. In another aspect, the monoclonal antibody comprises CDR sequences interposed between human and humanized framework sequences and further comprising a human germline framework sequence. In another aspect, the monoclonal antibody comprises CDR sequences interposed between human and humanized framework sequences wherein the framework sequence comprise at least one substitution at amino acid position 27, 30, 48, 67 or 78, where in the amino acid numbering is based on Kabat. In another aspect, the monoclonal antibody is combined with a cytotoxic agent, such that the antibody targets a RON expression protein and the RON-monoclonal antibody- and the cytotoxic agent are internalized into the cell. In another aspect, the monoclonal antibody is bound with a cytotoxic agent, such that the antibody targets a RON expression protein and the RON-monoclonal antibody- and the cytotoxic agent are internalized into the cell. In one aspect, the amino acid is at least one of SEQ ID NOS: 22, 24, 26, 28, 30, 32, 34, 36, 38 and 40. In one aspect, the antibody pairs at least one of SEQ ID NOS: 22, 24, 26, 28, 30, with at least one of SEQ ID NOS: 32, 34, 36, 38 and 40. In one aspect, nucleic acids are at least one of SEQ ID NOS: 21, 23, 25, 27, 29, 21, 33, 35, 37 and 39.

Yet another embodiment of the present invention includes an isolated nucleic acid comprising a nucleotide sequence encoding at least one on an immunoglobulin heavy chain variable region, or an immunoglobulin light chain variable region for a monoclonal antibody selected from Zt/g4-DM1, Zt/c1-DM1, Zt/64, 3F12, B9, 1G4, or Zt/f2. In another embodiment, the invention also includes an expression vector comprising a nucleic acid that expresses at least one of a monoclonal antibody selected from Zt/g4-DM1, Zt/c1-DM1, Zt/64, 3F12, B9, 1G4, or Zt/f2. Yet another embodiment includes a hybridoma cell selected from a Zt/g4-DM1, a Zt/c1-DM1, a Zt/64, a 3F12, a B9, a 1G4, or a Zt/f2 hybridoma cell that expressed an antibody that binds to human RON.

Another embodiment of the present invention includes a method of producing a polypeptide comprising an immunoglobulin heavy chain variable region or an immunoglobulin light chain variable region, the method comprising: growing the hybridoma cell outlined above under conditions so that the host cell expresses the polypeptide comprising the immunoglobulin heavy chain variable region or the immunoglobulin light chain variable region; and purifying the polypeptide comprising the immunoglobulin heavy chain variable region or the immunoglobulin light chain variable region.

Another embodiment of the present invention includes a method of producing an antibody that binds human RON or an antigen binding fragment of the antibody, the method comprising: growing the host cell of claim 9 under conditions so that the host cell expresses a polypeptide comprising the immunoglobulin heavy chain variable region and the immunoglobulin light chain variable region, thereby producing the antibody or the antigen-binding fragment of the antibody; and purifying the antibody or the antigen-binding fragment of the antibody.

Another embodiment of the present invention includes an isolated antibody that binds human RON, comprising an immunoglobulin heavy chain variable region and an immunoglobulin light chain variable region having at least a 95% homology to the sequences selected from the group consisting of the Heavy chains and Light chains of a monoclonal antibody selected from Zt/g4, DM1, Zt/c1-DM1, Zt/64, 3F12, B9, 1G4, or Zt/f2. In one aspect, immunoglobulin heavy chain variable region that comprises a CDR_{H1}; a CDR_{H2}; and a CDR_{H3} for a monoclonal antibody selected from Zt/g4-DM1, Zt/c1-DM1, Zt/64, 3F12, B9, 1G4, or Zt/f2; and an immunoglobulin light chain variable region that comprises: a CDR_{L1}; a CDR_{L2}; and a CDR_{L3} for a monoclonal antibody selected from Zt/g4-DM1, Zt/c1-DM1, Zt/64, 3F12, B9, 1G4, or Zt/f2. In another aspect, the CDR sequences are interposed between human and humanized framework sequences. In another aspect, the CDR sequences are interposed between human and humanized framework sequences further comprising a human germline framework sequence. In another aspect, the CDR sequences are interposed between human and humanized framework sequences wherein the framework sequence comprise at least one substitution at amino acid position 27, 30, 48, 67 or 78, where in the amino acid numbering is based on Kabat.

Yet another embodiment of the present invention includes a method of inhibiting or reducing proliferation of a tumor cell comprising exposing the cell to an effective amount of the antibody of claim 1 to inhibit or reduce proliferation of the tumor cell. In another embodiment, the invention includes a method of inhibiting or reducing tumor growth in a mammal, the method comprising exposing the mammal to an effective amount of the antibody of claim 1 to inhibit or reduce proliferation of the tumor.

Another embodiment includes a method of performing a clinical trial to evaluate a candidate drug believed to be useful in treating a disease condition related to at least one or RON overexpression, underexpression, kinase activity deregulation, RON transcript degradation, or RON degradation the method comprising: a) measuring the RON from tissue suspected of having a disease related to RON from a set of patients; b) administering a candidate drug to a first subset of the patients, and a placebo to a second subset of the patients; c) repeating step a) after the administration of the candidate drug or the placebo; and d) determining if the candidate drug reduces the number of cells that have the RON-related disease condition that is statistically significant as compared to any reduction occurring in the second subset of patients, wherein a statistically significant reduction indicates that the candidate drug is useful in treating said disease state. In one aspect, the candidate drug is an antibody that comprises at least one of a heavy chain or a light chain selected from Zt/g4-DM1, Zt/c1-DM1, Zt/64, 3F12, B9, 1G4, or Zt/f2.

Another embodiment of the present invention includes an isolated antibody that binds human RON, comprising an immunoglobulin heavy chain variable region and an immunoglobulin light chain variable region having at least a 95% homology to the amino acid sequences selected from the group consisting of: Heavy chains: SEQ ID NOS.: 2 or 4; and Light chains: SEQ ID NOS.: 6 or 8. In one aspect, the immunoglobulin heavy chain variable region comprises: a CDR_{H1} comprising an amino acid sequence selected from the group consisting of SEQ ID NOS.: 9 or 15; a CDR_{H2} comprising the amino acid sequence of SEQ ID NOS.: 10 or 16; a CDR_{H3} comprising the amino acid sequence of SEQ ID NOS.: 11 or 17; and an immunoglobulin light chain variable region comprises: a CDR_{L1} comprising the amino acid sequence of SEQ ID NOS.: 12 or 18; a CDR_{L2} comprising the amino acid sequence of SEQ ID NOS.: 13 or 19; and a CDR_{L3} comprising the amino acid sequence of SEQ ID NOS.: 14 or 20. In another aspect, the CDR sequences are interposed between human and humanized framework sequences. In another aspect, the CDR sequences are interposed between human and humanized framework sequences further comprising a human germline framework sequence. In another aspect, the CDR sequences are interposed between human and humanized framework sequences wherein the framework sequence comprise at least one substitution at amino acid position 27, 30, 48, 67 or 78, where in the amino acid numbering is based on Kabat.

Another embodiment of the present invention includes an isolated nucleic acid comprising a nucleotide sequence encoding at least one immunoglobulin heavy chain variable region SEQ ID NOS.: 1 or 3; or Light chains variable region SEQ ID NOS.: 5 or 7. Another embodiment of the present invention includes an expression vector comprising the nucleic acids from heavy chain variable region SEQ ID NOS.: 1 or 3; or light chains variable region SEQ ID NOS.: 5 or 7. Another embodiment of the present invention includes a host cell comprising the expression vector comprising heavy chain variable region SEQ ID NOS.: 1 or 3; or Light chains variable region SEQ ID NOS.: 5 or 7. Another embodiment of the present invention includes a method of producing a polypeptide comprising an immunoglobulin heavy chain variable region or an immunoglobulin light chain variable region, the method comprising: (a) growing the host cell of claim 9 under conditions so that the host cell expresses the polypeptide comprising the immunoglobulin heavy chain variable region or the immunoglobulin light chain variable region; and (b) purifying the polypeptide comprising the immunoglobulin heavy chain variable region or the immunoglobulin light chain variable region.

Another embodiment of the present invention includes a method of producing an antibody that binds human RON or an antigen binding fragment of the antibody, the method comprising: (a) growing the host cell of claim 29 under conditions so that the host cell expresses a polypeptide comprising the immunoglobulin heavy chain variable region and the immunoglobulin light chain variable region, thereby producing the antibody or the antigen-binding fragment of the antibody; and (b) purifying the antibody or the antigen-binding fragment of the antibody.

Another embodiment of the present invention includes an isolated antibody that binds human RON, comprising an immunoglobulin heavy chain variable region and an immunoglobulin light chain variable region having at least a 98% homology to the sequences selected from the group consisting of: Heavy chains: SEQ ID NOS.: 2 or 4; and Light chains: SEQ ID NOS.: 6 or 8. In one aspect, the immunoglobulin heavy chain variable region comprises: a CDR_{H1} comprising an amino acid sequence selected from the group consisting of SEQ ID NOS.: 9 or 15; a CDR_{H2} comprising the amino acid sequence of SEQ ID NOS.: 10 or 16; a CDR_{H3} comprising the amino acid sequence of SEQ ID NOS.: 11 or 17; and an immunoglobulin light chain variable region comprises: a CDR_{L1} comprising the amino acid sequence of SEQ ID NOS.: 12 or 18; a CDR_{L2} comprising the amino acid sequence of SEQ ID NOS.: 13 or 19; and a CDR_{L3} comprising the amino acid sequence of SEQ ID NOS.: 14 or 20. In another aspect, the CDR sequences are interposed between human and humanized framework sequences. In another aspect, the CDR sequences are interposed between human and humanized framework sequences further comprising a human germline framework sequence. In another aspect, the CDR sequences are interposed between human and humanized framework sequences wherein the framework sequence comprise at least one substitution at amino acid position 27, 30, 48, 67 or 78, where in the amino acid numbering is based on Kabat.

Another embodiment of the present invention includes a method of inhibiting or reducing proliferation of a tumor cell comprising exposing the cell to an effective amount of the antibody of claim 27 to inhibit or reduce proliferation of the tumor cell. Another embodiment of the present invention includes a method of inhibiting or reducing tumor growth in a mammal, the method comprising exposing the mammal to an effective amount of the antibody of claim 27 to inhibit or reduce proliferation of the tumor. Another embodiment of the present invention includes a method of treating cancer in a human patient, the method comprising administering an effective amount of the antibody of claim 27 to a mammal in need thereof.

Another embodiment of the present invention includes a method of evaluating a candidate drug believed to be useful in treating a disease condition related to at least one or RON overexpression, underexpression, kinase activity deregulation, RON transcript degradation, or RON degradation the method comprising: a) measuring the RON from tissue suspected of having a disease related to RON from a set of patients; b) administering a candidate drug to a first subset of the patients, and a placebo to a second subset of the patients; c) repeating step a) after the administration of the candidate drug or the placebo; and d) determining if the candidate drug reduces the number of cells that have the RON-related disease condition that is statistically significant as compared to any reduction occurring in the second subset of patients, wherein a statistically significant reduction indicates that the candidate drug is useful in treating said disease state. In one aspect, the candidate drug is an antibody, comprising an immunoglobulin heavy chain variable region and an immunoglobulin light chain variable region having at least a 98% homology to the sequences selected from the group consisting of: Heavy chains: SEQ ID NOS.: 2 or 4; and Light chains: SEQ ID NOS.: 6 or 8; or an antibody comprising the immunoglobulin heavy chain variable region comprises: a CDRH1 comprising an amino acid sequence selected from the group consisting of SEQ ID NOS.: 9 or 15; a CDRH2 comprising the amino acid sequence of SEQ ID NOS.: 10 or 16; a CDRH3 comprising the amino acid sequence of SEQ ID NOS.: 11 or 17; and an immunoglobulin light chain variable region comprises: a CDRL1 comprising the amino acid sequence of SEQ ID NOS.: 12 or 18; a CDR L2 comprising the amino acid sequence of SEQ ID NOS.: 13 or 19; and a CDR L3 comprising the amino acid sequence of SEQ ID NOS.: 14 or 20.

In another embodiment, the present invention includes an isolated nucleic acid having at least 95% sequence identity with nucleic acids comprising a sequence selected from at least one of SEQ ID NOS: 22, 24, 26, 28, 30, 32, 34, 36, 38 and 40. In another embodiment, the present invention includes a host cell comprising an isolated nucleic acid having at least 95%, 96, 97, 98, 99 or 100% sequence identity with nucleic acids comprising a sequence selected from at least one of SEQ ID NOS: 22, 24, 26, 28, 30, 32, 34, 36, 38 and 40. In another embodiment, the present invention includes an expression vector comprising an isolated nucleic acid having at least 95%, 96, 97, 98, 99 or 100% sequence identity with nucleic acids comprising a sequence selected from at least one of SEQ ID NOS: 22, 24, 26, 28, 30, 32, 34, 36, 38 and 40.

### DESCRIPTION OF THE DRAWINGS

For a more complete understanding of the features and advantages of the present invention, reference is now made to the detailed description of the invention along with the accompanying figures and in which:

FIGS. 1A to 1C show the generation and characterization of anti-RON ADC Zt/g4-DM1. FIG. 1A: Schematic representation of Zt/g4-DM1 structure: Zt/g4 is a mouse mAb specific to the RON sema domain (18). DM1 was conjugated to Zt/g4 by non-reducible thioether linkage (SMCC) through lysine residues in the antibody molecule. FIG. 1B: HIC analysis of the number of DM1 conjugated to Zt/g4: Individual Zt/g4-DM1s with different numbers of DM1 (0 to 8) are marked as P0 to P8. FIG. 1C: Stability of Zt/g4-DM1. Zt/g4-DM1 was kept at 37° C. for 30 days. Samples analyzed at different time-points with the average DARs were shown;

FIGS. 2A to 2F show the binding and induction of RON endocytosis by Zt/g4-DM1 in CRC cells. FIG. 2A: Levels of RON expression by different CRC cell lines: Five CRC cells lines (1×10⁶ cells/ml) in PBS were incubated at 4° C. with 5 μg/ml Zt/g4 for 60 min. Isotope matched mouse IgG was used as the control. Cell surface RON was quantitatively determined by the immunofluorescence assay using QIFKIT® reagents from DAKO (Carpentaria, Calif.) as detailed in Materials and Methods. FIG. 2B: Binding of Zt/g4-DM1 to human CRC cell lines: HCT116, HT29, and SW620 cells (1×10⁵ cells) were incubated with 5 μg Zt/g4-DM1 or Zt/c1-DM1. Free Zt/g4 and Zt/c1 was used as the control. Fluorescence intensity from individual samples was determined by flow cytometric analysis. FIG. 2C: Kinetic reduction of cell surface RON: HCT116, HT29, and SW620 cells (1×10⁶ cells per dish) were treated at 37° C. with 5 μg/ml of Zt/g4-DM1, collected at different time points, washed with acidic buffer to eliminate cell surface bound IgG, and then incubated with 1 μg/mL of anti-RON mAb 2F2 Immunofluorescence was analyzed by flow cytometer using FITC-coupled anti-mouse IgG Immunofluorescence from cells treated with Zt/g4-DM1 or Zt/c1-DM1 at 4° C. was set as 100%. Internalization efficiency was calculated as the time required to achieve 50% cell surface RON reduction. FIG. 2D: RON reduction analysis by Western blotting: Cellular proteins (50 μg per lane) from cells treated with 5 μg/ml of Zt/g4 or Zt/g4-DM1 for various times were separated in an 8% SDS-PAGE under reduced conditions and transferred to the membrane. RON was detected by rabbit anti-RON antibody followed enhanced chemiluminescent reagents. The same membrane was reprobed for actin as the loading control. FIG. 2E: Quantitative measurement of RON expression: The intensity of individual RON-β chains was determined by densitometric analysis. Internalization efficiency was calculated as the time required to achieve a 50% RON reduction. FIG. 2F: Immunofluorescent localization of cytoplasmic RON: HT29 cells (1×10⁵ cells per chamber) were treated at 4° C. or 37° C. with 5 μg/ml of Zt/g4-DM1 or Zt/c1-DM1 for 6 h followed by FITC-coupled anti-mouse IgG. After cell fixation, immunofluorescence was detected using the BK70 Olympus microscope equipped with a fluorescence apparatus. LAMP1 was used as a marker for protein cytoplasmic localization. DAPI was used to stain nuclear DNA;

FIGS. 3A to 3D show the effect of Zt/g4-DM1 on CRC cell cycle, survival, and death. FIG. 3A: Changes in cell cycles: Three CRC cell lines (1×10⁶ cells per dish) were treated at 37° C. with 5 μg/ml of Zt/g4-DM1 for various times, collected, stained with propidium iodide, and then analyzed by flow cytometer as previously described (32). FIG. 3B: Reduction of cell viability: Three CRC cell lines (5000 cells per well in a 96-well plate in triplicate) were treated with different amounts of Zt/g4-DM1 for 24, 48, and 72 h. Cell viability was determined by the MTS assay. FIG. 3C: Increased cell death: Cells were treated with different amount of Zt/g4-DM1 for 72 h. Morphological changes were observed under the Olympus BK-41 inverted microscope and photographed. Images showing cell death are presented. FIG. 3D: Cell death percentages were determined by the trypan blue exclusion method. The IC₅₀ values for cell viability or death at 72 h from individual groups were calculated using the GraphPad Prism 6 software. Results shown here are from one of three experiments with similar results;

FIGS. 4A to 4C show the therapeutic effect of a single dose Zt/g4-DM1 on CRC cell-derived tumors. Athymic nude mice (five mice per group) were subcutaneously inoculated with 5×10⁶ HCT116, HT29, and SW620 cells followed by injection of 20 mg/kg Zt/g4-DM1 through tail vein. FIG. 4A: Tumor growth from HT29-luc2 or HCT116-luc2 cells was determined by measuring average photon intensity (left panel). SW620-derived tumor growth was monitored by measuring tumor volume (FIG. 4B) (right panel). FIG. 4C: Tumor images with photon emission or caliper measurement at day 16 are presented. The scale from minimal to maximal is set at 300 to 35,000 photons per second. The percentages of inhibition were calculated from the average photon emission (for HT29 and HCT116 cells) or tumor volume (for SW620 cells). FIG. 4D: Individual tumors from different groups were weighed at day 28. The percentages of inhibition were calculated by a formula: (average tumor weight from Zt/g4-DM1 treated group/average tumor weight from control mice)×100%;

FIGS. 5A to 5F show the evaluation of different doses of Zt/g4-DM1 on tumor growth and RON expression. FIG. 5A: Effect of multi-dose of Zt/g4-DM1 on tumor growth was tested in HT29 cell-induced tumors. Tumor-bearing mice were treated with different doses of Zt/g4-DM1 every four days for a total of five injections ( [symbol] ). Tumor growth was determined by the average bioluminescence intensity. FIG. 5B: An IC₅₀ value based on the average bioluminescence intensity from individual groups at day 31 was calculated using GraphPad Prism 6 software. FIG. 5C: Bioluminescence images of individual tumors from each group at day 31 are shown. The percentages of inhibition were calculated from the average photon emission. The color scale from minimal to maximal is set at 300 to 35,000 photons per second. FIG. 5D: Individual tumors from different groups were collected and weighed at day 31, 35, and 43, respectively. The percentages of inhibition were calculated as detailed in FIG. 4C. FIG. 5E: Samples of HT29 cell-derived xenograft tumors from both control and 15 mg/kg Zt/g4-DM1-treated mice at day 31 were processed for histological examination. Analysis by H&E staining reveals cell death in different regions in Zt/g4-DM1-treated tumors but not in control samples. FIG. 5F: Western blot analysis of RON expression in tumors samples from both control and 15 mg/kg Zt/g4-DM1-treated mice. Densitometry analysis was performed to determine the levels of RON expression;

FIGS. 6A to 6C show the toxicity of Zt/g4-DM1 in vivo. Body weight was measured every four days during the period of Zt/g4-DM1 treatment. FIG. 6A: Effect of multiple doses of Zt/g4-DM1 on mouse body weight was determined by administration of Zt/g4-DM1 at 1, 3, 7, 10, 15 mg/kg every four day with a total of 5 injections. Mice were weighed and monitored for a total of 31 days. FIG. 6B: Effect of a single dose of Zt/g4 at 20 mg/kg on mouse body weight was determined using mice bearing HT29, HCT116, or SW620-derived tumors. Body weight was monitored up to 28 days. FIG. 6C: Effect of high doses of Zt/G4-DM1 on mouse body weight was analyzed by tail vein injection at 20, 40 and 60 mg/kg to Balb/c mice. Mice were euthanized at day 21. In all cases, the average body weight of mice before Zt/g4-DM1 injection was 19.8±3.6 grams (5 mice per group) and set as 100%;

FIG. 7 shows a schematic of the use of the monoclonal antibodies of the present invention;

FIG. 8 is a graph that shows that Zt/g4-DM1 induces cell surface RON reduction in pancreatic cancer cell lines;

FIG. 9 shows the Zt/g4-DM1-induced intracellular RON localization in pancreatic cancer cells;

FIGS. 10A to 11D are graphs that show the effect of Zt/g4-DM1 on pancreatic cancer cell cycle, viability, and apoptotic death;

FIGS. 11A to 11C are graphs that show a synergistic activity of Zt/g4-DM1 in combination with different chemotherapeutics; FIG. 11D includes graphs that show a synergistic activity of Zt/g4-MMAE in combination with Gemcitabine and viability of human pancreatic cancer cells; and FIG. 11E shows graphs that show the synergistic activity of Zt/g4-MMAE in combination with Oxaliplatin and viability of human pancreatic cancer cells;

FIG. 12 are graphs that show synergism between Zt/g4-DM1 and chemotherapeutics by isobolograms; and

FIG. 13 is a graph that shows the therapeutic effect of Zt/g4-DM1 at a single dose on xenograft growth of human PDACs.

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present inventors have developed a number of anti-RON mAbs that show biological and therapeutic effects in preclinical models. Anti-RON mAbs in conjugation with chemoagents are effective in the delivery of cytotoxic drugs to targeted killing of cancer cells. Understanding the MSP-RON signaling system can provide insight into the mechanisms of RON-mediated tumor pathogenesis, but also lead to the development of novel strategies to target or otherwise to use RON for effective cancer therapy.

The antibodies disclosed herein can be used to treat various forms of cancer, e.g., non-small cell lung cancer, breast, ovarian, prostate, cervical, colorectal, lung, pancreatic, gastric, and head and neck cancers. The cancer cells are exposed to a therapeutically effective amount of the antibody so as to inhibit or reduce proliferation of the cancer cell. In some embodiments, the antibodies inhibit cancer cell proliferation by at least 40%, 50%, 60%, 70%, 80%, 90%, 95%, 98%, 99%, or 100%.

The terms “a sequence essentially as set forth in SEQ ID NO. (#)”, “a sequence similar to”, “nucleotide sequence” and similar terms, with respect to nucleotides, refers to sequences that substantially correspond to any portion of the sequence identified herein as SEQ ID NO.: 1. These terms refer to synthetic as well as naturally-derived molecules and includes sequences that possess biologically, immunologically, experimentally, or otherwise functionally equivalent activity, for instance with respect to hybridization by nucleic acid segments, or the ability to encode all or portions of anti-RON antibodies. Naturally, these terms are meant to include information in such a sequence as specified by its linear order.

The term “homology” refers to the extent to which two nucleic acids are complementary. There may be partial or complete homology. A partially complementary sequence is one that at least partially inhibits a completely complementary sequence from hybridizing to a target nucleic acid and is referred to using the functional term “substantially homologous.” The degree or extent of hybridization may be examined using a hybridization or other assay (such as a competitive PCR assay) and is meant, as will be known to those of skill in the art, to include specific interaction even at low stringency.

An oligonucleotide sequence that is “substantially homologous” to the anti-RON antibodies of SEQ ID NO:#” is defined herein as an oligonucleotide sequence that exhibits greater than or equal to 75%, 80%, 85%, 90%, 91%, 92%, 93%, 94%, 95%, 96%, 97%, 98%, 99%, or 100% identity to the sequence of SEQ ID NO:# when sequences having a length of 100 bp or larger are compared. Generally, conservative amino acid substitutions will be used to modify the sequences within the listed percentages. Conservative amino acid substitutions are well-known in the art.

The term “gene” is used to refer to a functional protein, polypeptide or peptide-encoding unit. As will be understood by those in the art, this functional term includes at least partially genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

The term “vector” refers to a nucleic acid molecule(s) that transfer DNA segment(s) from one cell to another. The vector may be further defined as one designed to propagate specific sequences, or as an expression vector that includes a promoter operatively linked to the specific sequence, or one designed to cause such a promoter to be introduced. The vector may exist in a state independent of the host cell chromosome, or may be integrated into the host cell chromosome.

The terms “host cell”, “recombinant cell”, or “recombinant host” refer to cells that have been engineered to contain nucleic acid segments or altered segments, whether archeal, prokaryotic, or eukaryotic. Thus, engineered, or recombinant cells, are distinguishable from naturally occurring cells that do not contain recombinantly introduced genes.

The term “fusion protein” refers to a hybrid protein expressed by a nucleic acid molecule comprising nucleotide sequences of at least two genes. For example, a fusion protein can comprise at least part of a first and a second polypeptide fused with a polypeptide that binds an affinity matrix.

The term “antibody” encompasses polyclonal and monoclonal antibody preparations, as well as preparations including hybrid antibodies, altered antibodies, F(ab′)2 fragments, F(ab) fragments, Fv fragments, single domain antibodies, chimeric antibodies, humanized antibodies, and functional fragments thereof which exhibit immunological binding properties of the parent antibody molecule.

The term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)2, Fv, and other fragments that exhibit immunological binding properties of the parent monoclonal antibody molecule. In the case of the present invention, a number of hybridomas have been developed that, have unique binding properties with RON, e.g., they trigger specific internalization of RON into RON expressing cells, e.g., cancer cells. As used herein, the hybridoma and the antibody they produce use the same name, thus, the: Zt/g4-DM1, Zt/c1-DM1, Zt/64, 3F12, B9, 1G4, Zt/f2 hybridoma cells, produce the: Zt/g4-DM1, Zt/c1-DM1, Zt/64, 3F12, B9, 1G4, Zt/f2 monoclonal antibodies, respectively.

Methods of making monoclonal antibodies are known in the art. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the antigen may be conjugated to a bacterial toxoid, such as toxoid from diphtheria, tetanus, cholera, etc., in order to enhance the immunogenicity thereof.

Monoclonal antibodies are generally prepared using the method of Kohler and Milstein, Nature (1975) 256:495-497, or a modification thereof. Typically, a mouse, hamster, or rat is immunized. The spleen and/or large lymph nodes are is removed and dissociated into single cells. B-cells and/or dissociated spleen cells are then induced to fuse with myeloma cells to form hybridomas (typically cells that do not express endogenous antibody heavy and/or light chains), and are cultured in, e.g., a selective medium (e.g., hypoxanthine, aminopterin, thymidine medium, “HAT”). The resulting hybridomas are plated by limiting dilution and assayed for the production of antibodies that bind specifically to RON. The selected monoclonal antibody-secreting hybridomas are then cultured either in vitro (e.g., in tissue culture bottles or hollow fiber reactors), or in vivo (e.g., as ascites in mice).

The term “antibody fragment” refers to a portion of an antibody such as F(ab′)2, F(ab)2, Fab′, Fab, and the like. Regardless of structure, an antibody fragment binds with the same antigen that is recognized by the intact antibody. For example, an anti-RON monoclonal antibody fragment binds with an epitope of RON.

The term “antibody fragment” refers to a synthetic or a genetically engineered polypeptide that binds to a specific antigen, such as polypeptides that include light chain variable region(s), “Fv” fragments that include the variable regions of the heavy and light chains, recombinant single chain polypeptide molecules in which light and heavy variable regions are connected by a peptide linker (“scFv proteins”), and minimal recognition units that include the amino acid residues that mimic the hypervariable region.

The term Fab′ is defined herein as a polypeptide comprising a heterodimer of the variable domain and the first constant domain of an antibody heavy chain, plus the variable domain and constant domain of an antibody light chain, plus at least one additional amino acid residue at the carboxy terminus of the heavy chain C_{H}1 domain including one or more cysteine residues. F(ab′)₂ antibody fragments are pairs of Fab′ antibody fragments which are linked by a covalent bond(s). The Fab′ heavy chain may include a hinge region. This may be any desired hinge amino acid sequence. Alternatively the hinge may be entirely omitted in favor of a single cysteine residue or, a short (about 1-10 residues) cysteine-containing polypeptide. In certain applications, a common naturally occurring antibody hinge sequence (cysteine followed by two prolines and then another cysteine) is used; this sequence is found in the hinge of human IgG₁ molecules (E. A. Kabat, et al., Sequences of Proteins of Immunological Interest 3rd edition (National Institutes of Health, Bethesda, Md., 1987)). In other embodiments, the hinge region is selected from another desired antibody class or isotype. In certain preferred embodiments of this invention, the C-terminus of the C_{H}1 of Fab′ is fused to the sequence Cys X X. X preferably is Ala, although it may be any other residue such as Arg, Asp, or Pro. One or both X amino acid residues may be deleted.

The “hinge region” is the amino acid sequence located between C_{H}1 and C_{H}2 in native immunoglobulins or any sequence variant thereof. Analogous regions of other immunoglobulins will be employed, although it will be understood that the size and sequence of the hinge region may vary widely. For example, the hinge region of a human IgG₁ is only about 10 residues, whereas that of human IgG₃ is about 60 residues.

The term Fv is defined to be a covalently or noncovalently associated heavy and light chain heterodimer which does not contain constant domains.

The term Fv-SH or Fab′-SH is defined herein as a Fv or Fab′ polypeptide having a cysteinyl free thiol. The free thiol is in the hinge region, with the light and heavy chain cysteine residues that ordinarily participate in inter-chain bonding being present in their native form. In the most preferred embodiments of this invention, the Fab′-SH polypeptide composition is free of heterogeneous proteolytic degradation fragments and is substantially (greater than about 90 mole percent) free of Fab′ fragments wherein heavy and light chains have been reduced or otherwise derivatized so as not to be present in their native state, e.g. by the formation of aberrant disulfides or sulfhydryl addition products.

The term “chimeric antibody” refers to a recombinant protein that contains the variable domains and complementary determining regions derived from a rodent antibody, while the remainder of the antibody molecule is derived from a human antibody.

The term “humanized antibody” refers to an immunoglobulin amino acid sequence variant or fragment thereof that is capable of binding to a predetermined antigen and that includes an FR region having substantially the amino acid sequence of a human immunoglobulin and a complementarity determining regions (CDR) having substantially the amino acid sequence of a non-human immunoglobulin or a sequence engineered to bind to a preselected antigen. Humanizing an antibody is often referred to as “veneering” an antibody with the CDRs in the variable regions of the heavy, light chain or both.

As discussed herein, minor variations in the amino acid sequences of antibodies or immunoglobulin polypeptides are contemplated, e.g., providing that the variations in the amino acid sequence maintain at least 75%, more preferably at least 80%, 90%, 95%, 96%, 97%, 98%, 99%, and 100% homology to the human framework regions of the heavy and/or light chain variable domain. Specifically, in the present invention if the humanized antibody maintains at least 95%, 96%, 97%, 98%, 99%, or 100% homology to the non-CDR portions of the human variable domain and the constant domain, then the humanized antibody is considered to be fully humanized.

Certain variations in the amino acid sequences are considered conservative amino acid substitutions. Conservative substitutions are those between amino acids with similar side chains. Amino acids are generally divided into families: (1) non-polar: alanine, valine, leucine, isoleucine, proline, phenylalanine, methionine, tryptophan; (2) acidic: aspartate, glutamate; (3) basic: lysine, arginine, histidine; and (4) polar: lysine, asparagine, glutamine, cysteine, serine, threonine, tyrosine. Additional amino acid families include: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. Thus, it is reasonable to expect that a single replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework region. Whether an amino acid change results in a functional peptide is readily determined by assaying the specific activity of the polypeptide derivative. Fragments or analogs of antibodies or immunoglobulin molecules can be readily prepared by those of ordinary skill in the art and can substitutions of the amino- and carboxy-termini domains. Structural and functional domains can also be identified by comparison of the nucleotide and/or amino acid sequence data (as shown herein) and/or sequence databases. Computerized comparison methods can be used to identify sequence motifs or predicted protein conformation domains that occur in other proteins of known structure and/or function. Generally, conservative amino acid substitution will not substantially change the structural characteristics of the parent sequence (e.g., a replacement amino acid should not tend to break a helix that occurs in the parent sequence, or disrupt other types of secondary structure that characterizes the parent sequence).

The terms “cell” and “cell culture” are used interchangeably to refer to cell that are mostly but not always in a single cell suspension or attached to a plate or tissue, and include their progeny. The terms “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Different designations are will be clear from the contextually clear.

The terms “protein”, “polypeptide” or “peptide” refer to compounds comprising amino acids joined via peptide bonds and are used interchangeably.

The term “endogenous” refers to a substance the source of which is from within a cell. Endogenous substances are produced by the metabolic activity of a cell. Endogenous substances, however, may nevertheless be produced as a result of manipulation of cellular metabolism to, for example, make the cell express the gene encoding the substance.

The term “exogenous” refers to a substance the source of which is external to a cell. An exogenous substance may nevertheless be internalized by a cell by any one of a variety of metabolic or induced means known to those skilled in the art.

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “sequences” as used herein is used to refer to nucleotides or amino acids, whether natural or artificial, e.g., modified nucleic acids or amino acids. When describing “transcribed nucleic acids” those sequence regions located adjacent to the coding region on both the 5′, and 3′, ends such that the deoxyribonucleotide sequence corresponds to the length of the full-length mRNA for the protein as included. The term “gene” encompasses both cDNA and genomic forms of a gene. A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA I wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

The term “vector” is used in reference to nucleic acid molecules that transfer DNA segment(s) from one cell to another. The term “vehicle” is sometimes used interchangeably with “vector.” The term “vector” as used herein also includes expression vectors in reference to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome-binding site, often along with other sequences. Eukaryotic cells are known to utilize promoters, enhancers, and termination and polyadenylation signals.

The term a “pharmaceutically acceptable” refers to a component that is suitable for use with humans and/or animals without undue adverse side effects (such as toxicity, irritation, and allergic response) commensurate with a reasonable benefit/risk ratio.

The term “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, to shrink or not metastasize. The specific safe and effective amount or therapeutically effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

The term “pharmaceutical salts” refers to a salt for making an acid or base salts of a compounds. Examples of pharmaceutically acceptable salts include, but are not limited to, mineral or organic acid salts of basic residues such as amines; alkali or organic salts of acidic residues such as phenols. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The preferred phenolate salts are the alkaline earth metal salts, sodium, potassium or lithium.

The term “pharmaceutical carrier” refers to a pharmaceutically acceptable solvent, suspending agent or vehicle, for delivering the anti-RON antibodies, fragments thereof, and/or Antibody drug conjugates (ADCs), compound to the animal or human. The carrier may be liquid or solid and is selected with the planned manner of administration in mind. Liposomes are also a pharmaceutical carrier.

The term “cancer” refers to all types of cancer or neoplasm or malignant tumors found in mammals, including carcinomas and sarcomas. Examples of cancers are cancer of the brain, breast, cervix, colon, head & neck, kidney, lung, non-small cell lung, melanoma, mesothelioma, ovary, sarcoma, stomach, uterus and medulloblastoma.

The RON receptor tyrosine kinase is a potential drug target. Various types of tumors including breast and pancreatic cancers displayed aberrant RON expression featured by overexpression, isoform generation, and constitutive activation. Specific antibodies bind to RON on the surface of cancerous cells and cause RON internalization. This process is effective to deliver cytotoxic drugs for cancer treatment. Antibody drug conjugates (ADC) can be made as conjugates or fusion proteins using the present invention. The present inventors have recently developed a panel of anti-RON monoclonal antibodies (mAb) and prove that anti-RON mAbs are highly effect as drug delivery methods for potential cancer treatment.

ADCs are created by direct conjugation of highly toxic chemicals to oncogene-specific antibodies using advanced chemical linkers. The therapeutics suitable for chemical conjugation to antibodies are not regular anti-cancer chemoagents. Instead, they are highly toxic substances that cannot be directly injected into the patient body. The current drugs used for antibody chemical conjugations are monomethyl auristatin E, maytansine derivatives, and others. In July 2011, FDA approves brentuximab vedotin, an ADC that targets CD30 positive lymphomas, for leukemia treatment. Another ADC is trastuzumab conjugated with maytansine derivative for advanced breast cancer.

The RON receptor tyrosine kinase is a validated drug target for cancer therapy due to its high level expression in cancerous tissues. Currently, small molecules and therapeutic antibodies targeting RON are under preclinical and clinical trials. However, available results indicate that therapeutic effect was moderate due to the lack of strong addiction of RON signaling by tumor cells. Therefore, development of novel strategies to target RON is urgently needed.

The present invention includes a number of anti-RON mAbs ready for drug conjugation, preclinical efficacy study, in vivo toxicology evaluation, ADC distribution in vivo analysis, and targeted cancer profiling. The present inventors believe that by establishing this unique anti-RON ADC platform, it will help us to create a startup biotech company in Amarillo and to facilitate the collaboration/licensing with pharmaceutical companies to develop RON targeted ADC for cancer therapy.

The present inventors have developed a number of unique anti-RON mAbs that specifically recognize different epitopes on the RON extracellular domains/structures. The present inventors have proved that these mAbs rapidly cause RON internalization leading to effective drug uptake. These features position our anti-RON mAb in a unique situation for ADC development. Moreover, the present inventors have validated aberrant RON expression in various types of human cancer using these antibodies. These studies lead us to identify a panel of human cancers that are clinical targets of RON-mediated oncogenesis. Three major cancers with RON overexpression are colorectal, breast, and pancreatic cancers. Thus, the success in our anti-RON-directed ADC will have significant and broad market applications. Moreover, aberrant RON expression is also observed in erythroid leukemia, Hodgkin's lymphoma, and certain B-cell derived lymphomas, which add the additional clinical markets for the use of anti-RON ADC.

Accumulated evidence indicates that targeted RON inhibition by small molecule inhibitors or therapeutic antibodies only achieves moderate antitumor effect on various in vivo animal tumor xenograft models. Detailed analysis revealed that this is mainly due to the lack of strong RON signaling addiction by tumor cells. Also, tumor cells develop alternative signaling pathways to compensate RON-mediated inhibition of cell growth. However, targeting RON alone may not always be sufficient to control tumor growth and to show clinical significance. Moreover, it is highly urgent to develop novel strategies to target or otherwise to use RON for effective cancer therapy.

RON is overexpressed in colon, breast and pancreatic cancerous cells but remains at minimal levels in corresponding normal epithelial cells. This indicate that antibodies specific to RON can be utilized to carry cytotoxic drugs for targeted killing of RON expressing cancer cells and to improve the therapeutic index. To prove this concept, the present inventors have developed a panel of monoclonal antibodies specific to RON and used them conjugated with chemoagents to kill cancer cells. The present inventors have tested three types of cancer cells including regular colon, breast and pancreatic cancer cells, cancer cells under hypoxic conditions, and cancer stem cells. Results from these studies indicate that anti-RON mAb can induce a strong and rapid internalization of RON in cancerous cells and effectively delivers chemoagents for cytotoxicity.

Anti-RON mAbs and Therapeutic Properties.

The present inventors have produced a number of monoclonal antibodies specific to human RON, which have been validated to measure RON expression in cancerous tissues by immunohistochemical (IHC) staining and to test their anti-cancer activities in vitro and in vivo tumor models. More than twenty mAbs specific to the RON extracellular domains and tested their biochemical and biological properties.

Three Types of Anti-RON mAbs:

Using an advanced living cell immunization method, the present inventors were able to generate mAbs specific only to the RON extracellular domains. Using flow cytometer in conjunction with biological assays, the present inventors characterize our anti-RON mAbs for their specificity and sensitivity. Currently, these anti-RON mAbs have been shown to be highly sensitive and specific to human RON. This is based on direct binding, ELISA, IHC, Western blotting and other biochemical and biological assays. Moreover, based on their activities upon binding to RON, the present inventors were able to classify the anti-RON mAb into three categories. (1) antibodies that bind to RON and cause transient RON phosphorylation. This type of anti-RON mAbs is considered as agnostic antibodies. The representatives are Zt/g4, Zt/c1, Zt/c9, Zt/f1, and Zt/H12. (2) anti-RON mAbs are those that binds to RON but did not activate RON. The typical examples are Zt/g9 and Zt/c8. (3) Anti-RON mAbs are those that bind to RON and inhibit RON activation and signaling. One example is Zt/f2. The present inventors considered this type of mAb having therapeutic potentials.

Agonistic Anti-RON mAbs Induces Rapid RON Endocytosis.

During characterization of anti-RON mAbs, the present inventors discovered that agonistic anti-RON mAb such as Zt/g4 and Zt/c1 binds to RON and cause rapid and significant amount of cell surface RON internalization in cancerous cells (a process known as antibody-induced receptor endocytosis). The effect is highly efficient, within 24 h after addition of anti-RON mAb Zt/g4 at 10 μg/ml/1×10⁶ cancer cells, almost all cell surface RON is internalized. More interestingly, the endocytosis will interfere with intracellular RON synthesis, leading to the absence of RON expression in cancerous cells for up to 72 h in culture. Using various biochemical/biological assays, the present inventors demonstrated that Zt/g4 induced RON phosphorylation is required for RON endocytosis. Fab fragments that fail to cause RON activation cannot induce RON endocytosis. In light of these findings, the present inventors conclude that anti-RON mAb-induced RON endocytosis can be used as a pharmaceutical means for targeted drug delivery.

Anti-RON mAb Directed Deliveries of Chemoagents for Enhanced Cancer Cell Killing.

To demonstrate that antibody-directed RON endocytosis for efficient drug delivery, the present inventors used an advanced immunoliposome technology known as stealth immunoliposome to prepare Zt/g4 or Zt/c1-immunoliposomes loaded with doxorubicin (Zt/g4-Dox-IL or Zt/c1-Dox-IL). Using various controls, the present inventors demonstrated that Zt/g4 or Zt/c1-dox-IL bids specifically to RON expressing cancer cells and cause a rapid endocytosis of RON, which leads to delivery of Dox into the cytoplasm of cancerous cells. The cytotoxic efficacy was significant improved compared to cells that are resistant to free drugs. Moreover, the present inventors tested the therapeutic index of Zt/g4 or Zt/c1-Dox-IL in cancer cells under three different conditions including normoxia, hypoxia, and stemness. Finally, the present inventors used Zt/g4 or Zt/c1-Dox-IL in different types of cancer cells such as colon, breast, and pancreatic cancer cells, the improved cytotoxic activities were demonstrated in all cell line tested. Thus, our results demonstrated that the use of anti-RON mAbs to delivery of cytotoxic agents is an effective in improving anticancer efficacy of common chemoagents. Also, these observations lay the foundation for development of ADC for potential clinical application.

Anti-RON mAb Zt/f2 is a Therapeutic Antibody Directly Inhibiting Tumor Growth In Vivo.

Zt/f2 is a mouse IgG2a mAb that is highly specific and sensitive to human RON and its oncogenic variants such as RON160 (ED50=2.3 nmol/L). Receptor binding studies revealed that Zt/f2 interacts with an epitope(s) located in a 49 amino acid sequence coded by exon 11 in the RON β-chain extracellular sequences. This sequence is critical in regulating RON maturation and phosphorylation. Zt/f2 did not compete with ligand macrophage-stimulating protein for binding to RON; however, its engagement effectively induced RON internalization, which diminishes RON expression and impairs downstream signaling activation. These biochemical features provide the cellular basis for the use of Zt/f2 to inhibit tumor growth in animal model. Repeated administration of Zt/f2 as a single agent into Balb/c mice results in partial inhibition of tumor growth caused by transformed NIH-3T3 cells expressing oncogenic RON160. Colon cancer HT-29 cell-mediated tumor growth in athymic nude mice also was attenuated following Zt/f2 treatment. In both cases, ˜50% inhibition of tumor growth as measured by tumor volume was achieved. Moreover, Zt/f2 in combination with 5-fluorouracil showed an enhanced inhibition effect of ˜80% on HT-29 cell-mediated tumor growth in vivo. The present inventors conclude that Zt/f2 is a potential therapeutic mAb capable of inhibiting RON-mediated oncogenesis by colon cancer cells in animal models. The inhibitory effect of Zt/f2 in vivo in combination with chemoagent 5-fluorouracil could represent a novel strategy for future colon cancer therapy.

Biotherapeutic Platform for anti-RON mAb ADC.

An anti-RON mAb based biotherapeutic platform can be established that facilitates development and licensing of our unique anti-RON mAb to pharmaceutical and biotechnology companies for development of anti-RON ADC for clinical application.

Components: The anti-RON mAb based therapeutic platform can include one or more of the following components:

(1) Improved living cell immunization technology: This technique uses living cells overexpressing RON and its variants as the immunogens to ensure that hybridomas produce anti-RON specific antibodies recognizing only RON extracellular domains. Further improvement will include structural analysis of RON extracellular domains, which should help us to produce antibodies with improved activity for RON endocytosis.

(2) Anti-RON mAb humanization technology: Humanize selected anti-RON mAbs for future ADC development. The present inventors will conduct anti-RON mAb mRNA isolation and sequence analysis. The unique antigen binding sequences can be grafted into human IgG1 using commercially available methods.

(3) Antibody characterization technology: The present inventors have produced more than twenty anti-RON mAbs, which need to be fully characterized for their potentials as ADC suitable anti-RON mAbs. A series of standardized assay/methods can be used to characterize these anti-RON mAbs and to finalize their status for potential ADC development. Examples of assays include: binding domain/region and specificity, binding sensitivity & affinity, RON endocytosis inducing capability, and drug uptake efficacy.

(4) Anti-Ron mAb Production/Characterization/Profiling. The present inventors can also use the antibodies for additional development and implementation of standardized procedures for immunization using living cells overexpressing RON and RON variants. The purpose is to select the best domain/region for production of anti-RON mAb with high specificity, sensitivity, and capable of inducing robotic RON endocytosis for drug delivery. Moreover, the present inventors can also program assays/methods to speed up the characterization and profiling procedures to select anti-RON mAbs for humanization and additional ADC development.

(5) Anti-RON mAb Humanization and Preclinical Evaluation. For anti-RON mAb humanization, mRNA sequences of selected anti-RON mAb such as Zt/f2 and Zt/g4 can be used to identify additional antigen binding sequences. Selection of best region for sequence grafting to generate humanized anti-RON mAb Zt/f1 and Zt/g4 can be used in conjunction with characterization/profiling, using the inventors' standardized assay/methods to evaluate the antibody specificity and sensitivity. The drug-conjugated anti-RON mAbs can be evaluated in various preclinical models for therapeutic efficacy.

ADC, the second generation of therapeutics for targeted cancer therapy, has been advanced rapidly for the last several years due to the success in chemical linking technology. Significantly, the limited efficacy of the first generation of therapeutic antibodies against cancer has called for novel strategies for effective cancer therapy. Currently, more than twenty ADC is under clinical trials with promising results. RON is a valid drug target. The present inventors have generated more than twenty anti-RON mAbs for direct cancer treatment and used them for drug delivery.

Efficacy of Anti-RON Antibody Zt/g4-Drug Maytansinoid Conjugation (Anti-RON ADC) as a Novel Therapeutics for Targeted Colorectal Cancer Therapy. The receptor tyrosine kinase RON is critical in epithelial tumorigenesis and a drug target for cancer therapy. Here we report the development and therapeutic efficacy of a novel anti-RON antibody Zt/g4-maytansinoid (DM1) conjugates for targeted colorectal cancer (CRC) therapy.

Monoclonal antibody Zt/g4 (IgG1α/κ) was conjugated to DM1 via thioether linkage to form Zt/g4-DM1 with a drug-antibody ratio of 4:1. CRC cell lines expressing different levels of RON were tested in vitro to determine Zt/g4-DM1-induced RON endocytosis, cell cycle arrest, and cytotoxicity. Efficacy of Zt/g4-DM1 in vivo was evaluated in mouse xenograft CRC tumor model.

Zt/g4-DM1 rapidly induced RON endocytosis, arrested cell cycle at G2/M phase, reduced cell viability, and caused massive cell death within 72 h. In mouse xenograft CRC models, Zt/g4-DM1 at a single dose of 20 mg/kg body weight effectively delayed CRC cell-mediated tumor growth up to 20 days. In a multiple dose-ranging study with a five injection regimen, Zt/g4-DM1 inhibited more than 90% tumor growth at doses of 7, 10, and 15 mg/kg body weight. The minimal dose achieving 50% of tumor inhibition was ˜5.0 mg/kg. The prepared Zt/g4-DM1 is stable at 37° C. for up to 30 days. At 60 mg/kg, Zt/g4-DM1 had a moderate toxicity in vivo with an average of 12% reduction in mouse body weight.

It was found that Zt/g4-DM1 is highly effective in targeted inhibition of CRC cell-derived tumor growth in mouse xenograft models. This work provides the basis for development of humanized Zt/g4-DM1 for RON-targeted CRC therapy in the future.

Aberrant RON expression is a pathogenic factor contributing to epithelial tumorigenesis. However, therapeutic antibodies or tyrosine kinase inhibitors targeting RON for cancer therapy have shown very limited efficacy. Thus, there is a need to develop RON-targeted therapeutics with improved efficacy. Novel therapeutics in the form of anti-RON antibody Zt/g4-drug maytansinoid conjugates (Zt/g4-DM1) for targeted cancer therapy are described herein. It was found that Zt/g4-DM1 retains its intrinsic activity that induces RON endocytosis, resulting in cell cycle arrest, reduced cell viability, and massive cell death. In mouse xenograft tumor models, Zt/g4-DM1 displays a strong efficacy and a long-lasting effect on colorectal cancer cell-derived tumors with a favorable safety profile. Thus, targeted CRC therapy can be significantly improved by anti-RON antibody-drug conjugates, which have broad implications for treatment of various types of cancers. In this sense, Zt/g4-DM1 represents a novel antibody-drug conjugate.

The RON receptor tyrosine kinase, a member of the MET proto-oncogene family (1,2), has been implicated in epithelial tumorigenesis (3). Overexpression of RON exists in various primary tumors including colorectal, breast, and pancreatic cancers (4-10). In colorectal cancers (CRC), RON is overexpressed in more than 50% of cases (4,5). Aberrant RON expression also results in generation of oncogenic and constitutively active RON variants such as RONΔ160 (3,5). The consequence of these abnormalities is the activation of various intracellular signaling pathways that facilitate CRC cell growth, invasion, and chemoresistance (3). Overexpression of RON in CRC also has prognostic value in predicting patient survival and clinical outcomes (11). Thus, aberrant RON expression is a pathogenic feature in CRC cells, which contributes to tumorigenic phenotype and malignant progression (3-5, 11-13).

The high frequency of CRC RON overexpression and the dependency of CRC cells on RON signaling for growth provide the rationale to target RON for therapy. Tyrosine kinase inhibitors (TKI) such as foretinib (14), BMS-777607 (15), and MK-2461 (16) that target RON and MET are currently under clinical trials (www.clinicaltrials.gov). Therapeutic monoclonal antibodies (TMA) specific to RON such as IMC-41A10, narnatumab (clinical trial ID: NCT01119456), and Zt/f2 also have been evaluated in preclinical models (17,18). Results indicate that targeted inhibition of RON has a therapeutic effect on tumors mediated by colon, breast, and pancreatic cancer cells in animal models (17-19). However, efficacy is limited to only about 40-50% (17-19). Complete inhibition of tumor growth by a single RON-targeted TKI or TMA has not been observed (14-19). Thus, there is an urgent need to develop and improve the efficacy of RON targeted-therapeutics.

One highly attractive strategy to enhance efficacy is to target RON for cytotoxic drug delivery. First, RON is preferentially expressed in cancer cells with minimal expression in corresponding normal epithelial cells (4-10). Also, RON is not expressed in fibroblasts, endothelial cells, and blood leukocytes (1, 4, 7, 20). Such expression pattern is crucial for achieving the maximal drug delivery with manageable safety profiles. Second, RON-specific monoclonal antibodies (mAb) such as Zt/g4 and Zt/f2 rapidly induce RON internalization by cancer cells (21-24). This process requires a transient RON phosphorylation, which is essential for receptor endocytosis (21-24). Finally, anti-RON mAb-directed drug delivery, which exerts increased cytotoxicity against cancer cells, has been proven in experimental CRC therapy (21-24). Considering the advanced technology used in antibody-drug conjugates (ADC) for targeted cancer therapy (25), the development of anti-RON ADC is a promising strategy for RON-targeted therapy. This approach should also overcome the shortcomings in TKI- or TMA-targeted therapies that depend on RON signaling for the growth and survival of cancer cells.

The present study evaluates a novel anti-RON ADC for CRC therapy. It was found that RON-directed delivery of highly potent drug in the form of ADC was effective in inhibiting tumor growth in mouse xenograft CRC models. ADC is a combination of target-specific antibody, highly potent compound, versatile chemical linker, and controlled drug payload. The development of anti-RON ADC provides a rational approach to evaluate the efficacy of RON-targeted therapy. To this end, the inventors selected the mouse mAb Zt/g4, which is highly specific to the RON extracellular sequences as the drug carrier. Zt/g4 was conjugated to maytansinoid known as DM1 through non-reducible thioether linkage (24). The efficacy of anti-RON Zt/g4 ADC was evaluated using in vitro and in vivo models.

Cell Lines and Reagents: CRC cell lines DLD1, LoVo, HCT116, HT29, and SW620 were from American Type Cell Culture (Manassas, Va.) and authenticated in 2010 with cytogenesis. HT29-luc2 and HCT116-luc2 cells expressing the firefly luciferase gene-2 were from Perkin Elmer (Waltham, Mass.) and authenticated in 2011 with DNA profiling and cytogenesis. Mouse anti-RON mAbs Zt/g4, Zt/c1 and rabbit IgG antibody to the RON C-terminal peptide were used as previously described (2). Goat anti-mouse IgG labeled with fluorescein isothiocyanate (FITC) or rhodamine was from Jackson ImmunoResearch (West Grove, Pa.). Maytansinoid (DM1) and N-succinimidyl-4-[maleimidomethyl]-cyclohexane carboxylate (SMCC) were from Concortis (San Diego, Calif.).

Conjugation of anti-RON mAb with DM1 through thioether linkage: Conjugation was performed according to a protocol to achieve a drug-antibody ratio (DAR) at 4:1 (26, 29, 30). Briefly, Zt/g4 at 10 mg/ml was mixed with 10 mM SMCC-DM1 in a conjugation buffer to form Zt/g4-SMCC-DM1 (designated as Zt/g4-DM1). The anti-RON mAb Zt/c1 also was conjugated with SMCC-DM1 to form Zt/c1-DM1. We also prepared the control ADC by conjugating normal mouse IgG (CmIgG) with SMCC-DM1 to form CmIgG-DM1 as described above. All conjugates were purified using a PC10 Sephadex G25 column, sterilized through a 0.22 μM filter, and stored at 4° C.

Analysis of Zt/g4-DM1 conjugation and its stability: The conjugation of DM1 to Zt/g4 was verified by hydrophobic interaction chromatography (HIC) using a Varian Prostar 210 Quaternary HPLC system coupled with a TSK butyl-NPR 4.6×3.5 column (Tosoh Biosciences (Prussia, Pa.) (31). The average DARs were calculated from the integrated areas of the DAR species. This method also was used to determine the stability of Zt/g4-DM1 at 37° C.

Assay for cell surface RON expression: Cell surface RON was quantitatively determined by the immunofluorescence assay using QIFKIT® reagents from DAKO (Carpentaria, Calif.). Cells (1×10⁶ cells per ml in PBS) were treated with Zt/g4 at saturating concentrations followed by incubation in parallel with the QIFIKIT® beads and goat F(ab′)₂ F0479. After establishing a calibration curve, the number of RON receptor on the cell surface was then determined by interpolation following the manufacturer's instruction.

Western blot analysis of RON expression: Cellular proteins (50 μg per sample) were separated in an 8% SDS-PAGE under reduced conditions. Western blotting of RON expression was performed as previously described (2). Membranes also were reprobed with anti-actin antibody to ensure equal sample loading.

Detection of internalized RON: Cells at 1×10⁵ cells per well in a 6-well plate were treated with 5 μg/ml Zt/g4 or Zt/g4-DM1 for various times followed by goat anti-mouse IgG coupled with FITC or rhodamine. Nuclear DNAs were stained with 4′,6-diamidino-2-phenylindole Immunofluorescence was observed under an Olympus BK71 microscope equipped with DUS/fluorescent apparatus as previously described (32).

Cell viability and death assays: Cell viability 72 h after Zt/g4-DM1 treatment was determined by the MTT assay (22). Viable or dead cells were determined by the trypan blue exclusion assay. A total of 900 cells were counted from three individual wells to reach the percentages of dead cells.

Analysis of cell cycle: HT29, HCT116, and SW620 cells (1×10⁶ cells per dish) were incubated at 37° C. with 5 μg/ml Zt/g4-DM1 for 24 h, labeled with propidium iodide, and then analyzed by an Accuri Flow Cytometer. Cell cycle changes were determined by measuring DNA contents as previously described (32).

Mouse xenograft CRC model and anti-RON ADC treatment: All mice studies were approved by the institutional animal care committee. Female athymic nude mice at 6 weeks of age (Taconic, Cranbury, N.J.) were injected with 5×10⁶ HT29-Luc2, HCT116-luc2, or SW620 cells in the subcutaneous space of the right flank as previously described (18,33). Mice were randomized into different groups (five mice per group). Treatment began when all tumors had reached an average bioluminescence of ˜1×10⁷ (for HT29- and HCT116-luc2 cells) or a mean tumor volume of ˜100 mm³ (for SW620 cells). The single-dose group received a tail vein injection of 20 mg/kg Zt/g4-DM1 in 0.1 ml PBS followed by observation for 28 days. The multi-dose study was performed by treating mice with Zt/g4-DM1 at 1, 3, 7, 10, and 15 mg/kg every four days for a total of five injections. Bioluminescence from individual tumors was measured every four days using Caliper IVIS image system (PerkinElmer). Tumor volumes from SW620-derived tumors were measured according to a formula: V=pi/6×1.58×(length×width)^{3/2} (18,33). Animals were euthanized when tumor volumes exceeded 2000 mm³ or if tumors became necrotic or ulcerated through the skin.

In vivo toxicity studies: Acute toxicity with maximum tolerated dose was determined in Balb/C mice (four mice per dose) by a single tail vein injection of Zt/g4-DM1 at 20, 40, and 60 mg/kg body weight. Toxicity associated with different therapeutic doses was evaluated in athymic nude mice bearing HT29 tumor xenograft (five mice per dose). Mice were observed for about 30 days. Toxicity was assessed by observing mouse behavior, weight loss, and survival.

Statistical analysis: GraphPad Prism 6 software was used for statistical analysis. Results are shown as mean±SD. The data between control and experimental groups were compared using Student t test. Statistical differences at p<0.05 were considered significant.

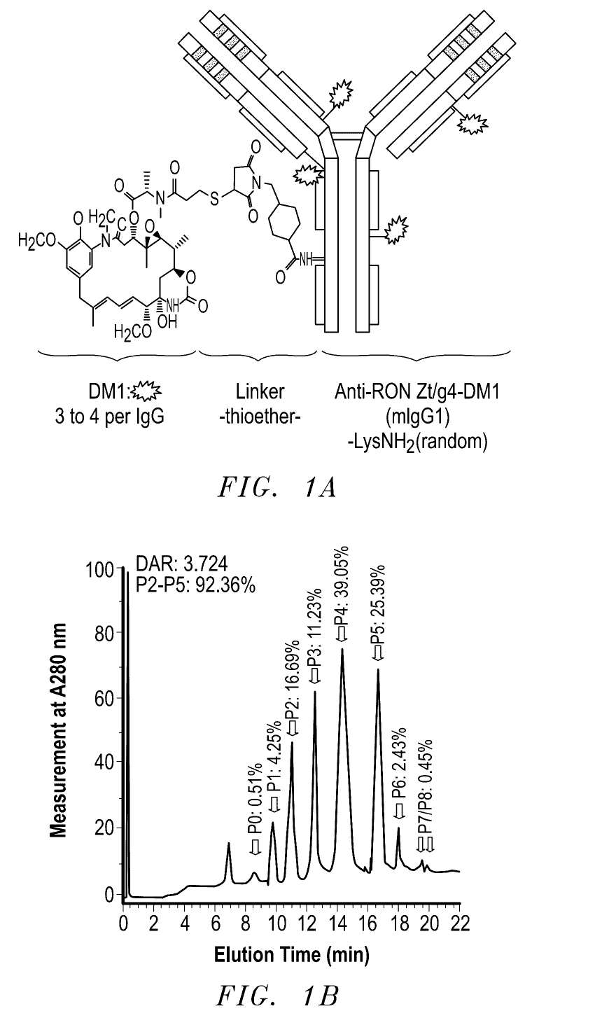

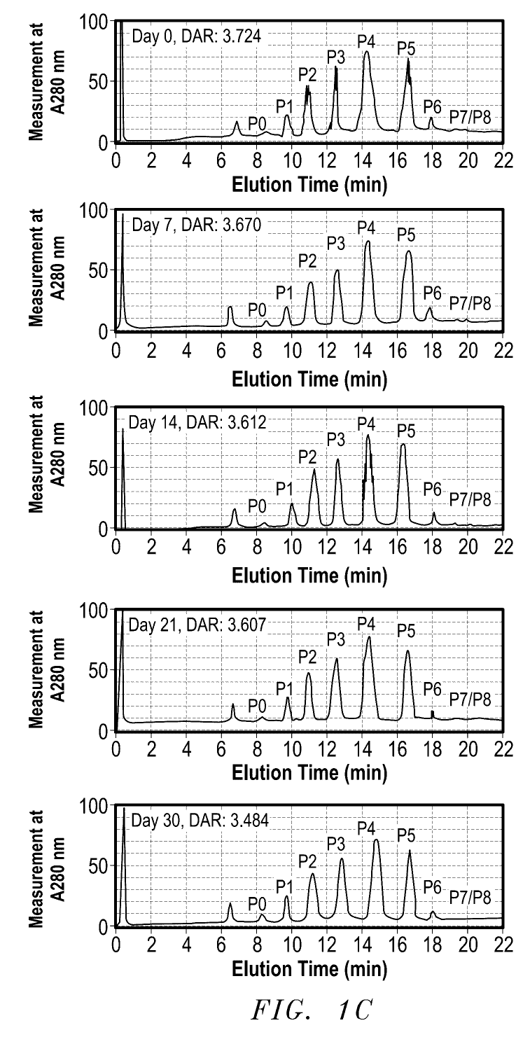

Characterization of anti-RON ADC Zt/g4-DM1: Zt/g4 was selected as a lead ADC candidate due to its ability to induce RON internalization in various cancer cells (data not shown) (21-23,28). Zt/g4 only recognizes human RON but not mouse RON homologue (28) and by itself has no tumor agonistic effect in vivo (18). Structures of Zt/g4-DM1 are shown in FIG. 1A. A total of 250 μg Zt/g4 was conjugated to DM1 with conditions to achieve an average DAR of 4:1. Our selection of this ratio was based on published observations of trastuzumab-emtansine (T-DM1) in which one IgG molecule coupling with four DM1 molecules achieves maximal therapeutic efficacy (26,27). HIC analysis revealed average DARs of Zt/g4-DM1 at 3.724 (FIG. 1B). The percentages of conjugates with different DARs from the integrated areas of the conjugates also were determined (FIG. 1B and data not shown). The major peak accounting for 39.05% was peak 4 with a DAR of 4:1. The prepared Zt/g4-DM1 with DARs at 5:1, 4:1, 3:1, and 2:1 accounted for more than 92% of the total conjugates. DARs for Zt/c1-DM1 and CmIg-DM1 were 3.91 and 4.01, respectively.

The stability of Zt/g4-DM1 was determined by incubating the conjugates in vitro at 37° C. for 30 days. DAR changes were measured by HIC from different time-points. Zt/g4-DM1 appears to be stable at 37° C. for up to 30 days (FIG. 1C and data not shown). At day 30, it has an average DAR of 3.484, which represents only a 6.4% reduction from the DAR of 3.724 at day 0. The major changes appeared to be peak 4 and peak 5, which were reduced from 39.05% to 32.72% for peak 4 and 25.39% to 20.06% for peak 5, respectively. Thus, the prepared Zt/g4-DM1 has a suitable DAR and is relatively stable at 37° C.

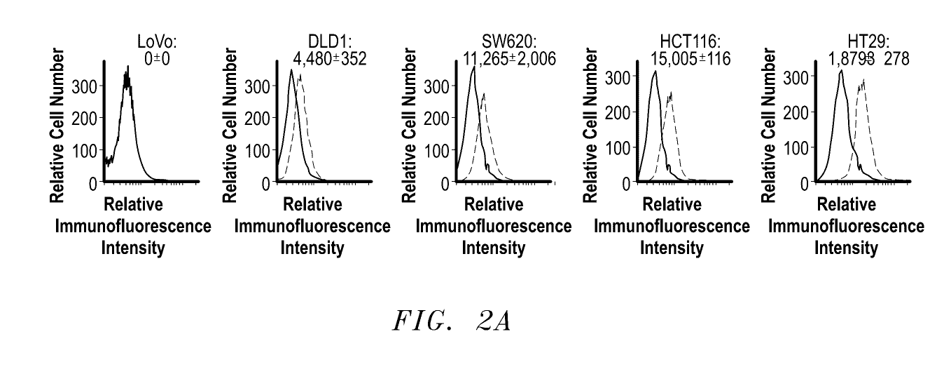

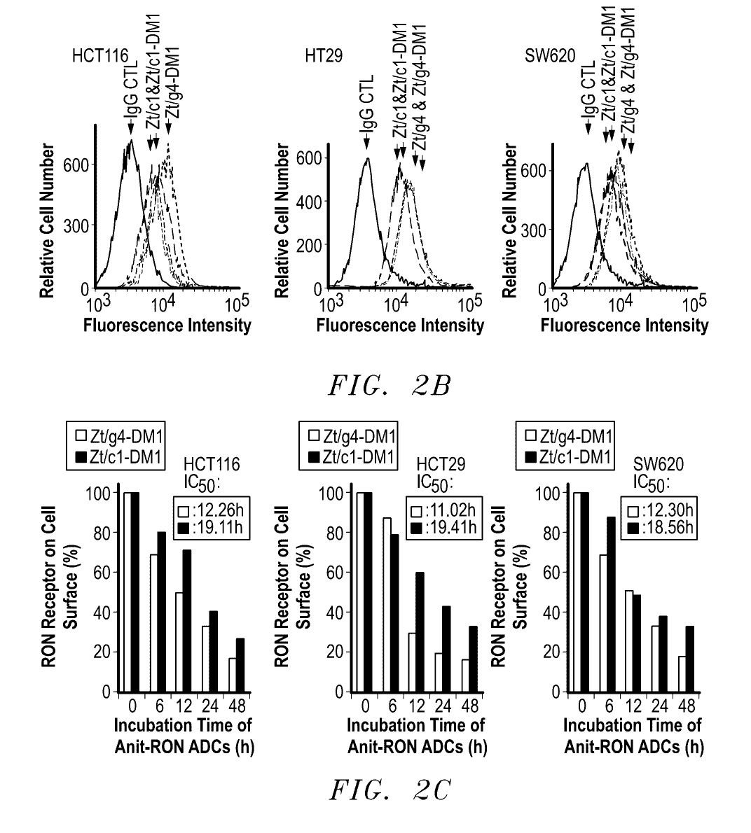

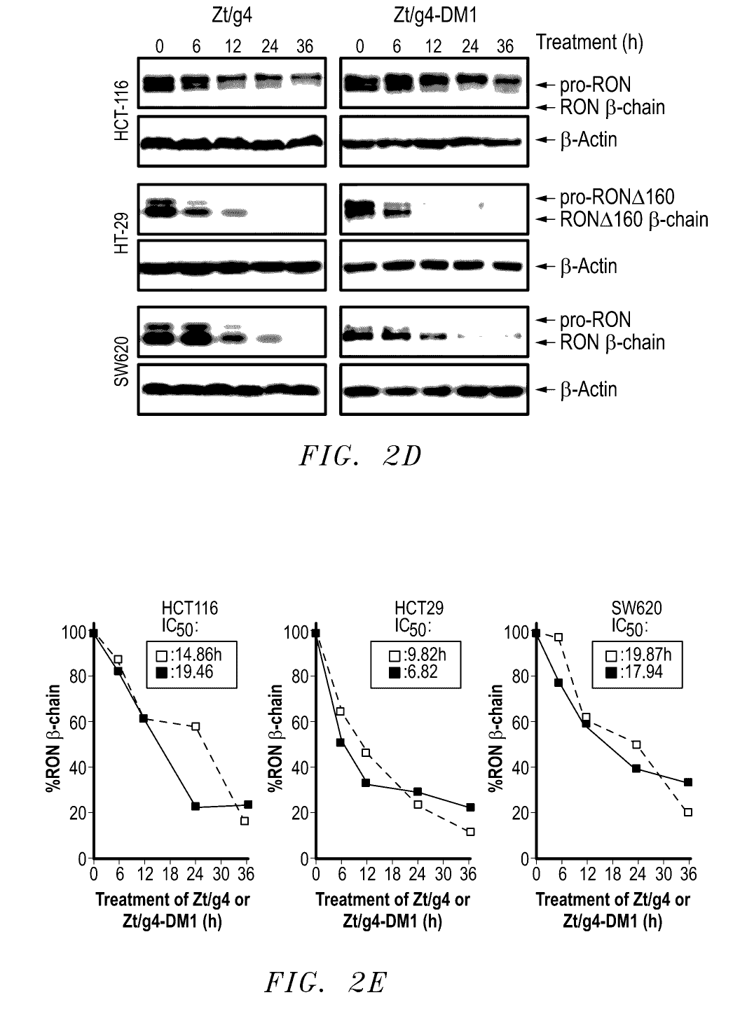

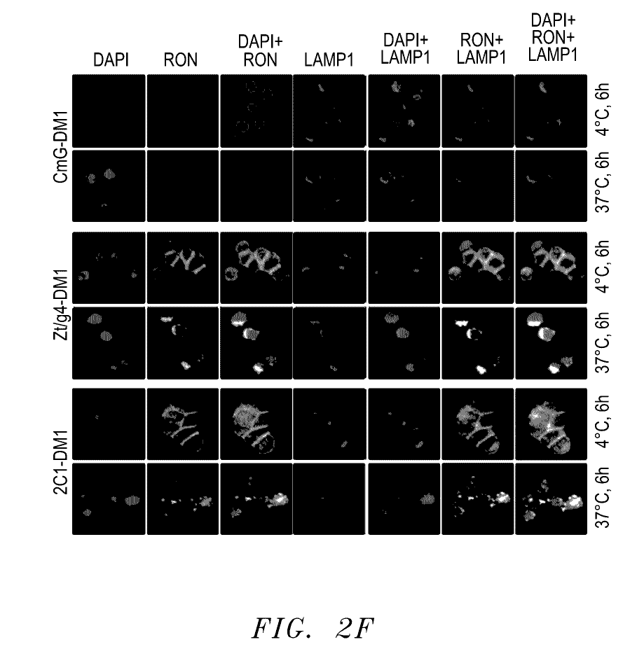

Effect of Zt/g4-DM1 on induction of RON endocytosis by CRC cells: We selected CRC cell lines LoVo, DLD1, HT29, HCT116, and SW620 expressing variable levels of RON as the cellular model (5,13). RON signaling is implicated in growth, survival, and invasion in HT29, HCT116 and SW620 cells (data not shown). The number of RON receptors expressed on CRC cell surfaces was determined by the QIFKIT® fluorescence-based quantitative method (FIG. 2A). The calculated RON molecules on the surface of a single CRC cell was 18,793±278 for HT29, 15,005±115.62 for HCT116, and 11,265±2,006 for SW620 cells, respectively. DLD1 has about 4,480±347 specific-binding sites per cell. Specific binding was not observed in LoVo cells. The binding capacity of Zt/g4-DM1 to RON was determined by flow cytometric analysis. No difference in binding intensity between free Zt/g4 and Zt/g4-DM1 in all three CRC cell lines tested (FIG. 2B) was found. Thus, the conjugation did not impair the Zt/g4 binding capability.

Zt/g4-DM1-induced RON endocytosis was studied, which is a process essential for delivering DM1 into CRC cells. Zt/g4-DM1 causes a progressive reduction of cell surface RON in a time-dependent manner in all three CRC cell lines tested (FIG. 2C). Less than 20% of RON remained on the cell surface after a 48 hour treatment. The time required for Zt/g4-DM1 to induce 50% RON reduction (internalization efficacy) was at 12.26 h, 11.02 h, and 12.30 h for HCT116, HT29, and SW620 cells, respectively. In contrast, the time required for Zt/c1-DM1-induced 50% RON reduction in HCT116, HT29, and SW620 was at 19.11 h, 19.41 h, and 18.65 h, respectively. Thus, Zt/g4-DM1 is more efficient and potent in induction of RON endocytosis.

Western blotting was performed to verify the effect of Zt/g4-DM1 on RON expression (FIG. 2D). Both pro-RON and mature RON (indicated by RON-β chain) were progressively reduced in all three CRC cell lines tested. Zt/g4-DM1 was effective in reducing mature RON expression, which resides on the cell surface. Less than 20% of the RON-β chain was detected 36 h after Zt/g4-DM1 treatment. The kinetic reduction of mature RON was quite different among three cell lines (FIG. 2E). However, the patterns of Zt/g4-DM1-induced RON reduction were comparable to those of free Zt/g4-induced RON reduction, suggesting that the conjugation does not impair the ability of Zt/g4-DM1 to induce RON endocytosis.

Zt/g4-DM1-induced RON endocytosis was confirmed by immunofluorescence analysis of cytoplasmic RON using HT29 cells as the model (FIG. 2F). Cells stained for lysosomal-associated membrane protein 1 (LAMP1) were used as a marker for co-localization of internalized RON. At 4° C., RON is detected on the cell surface. The intracellular localization of internalized RON occurred at 37° C. after Zt/g4-DM1 treatment. Also, the cytoplasmic RON was co-localized with LAMP1 in HT29 cells, indicating that internalized RON resides within lysosomes. In contrast, RON endocytosis was minimal in cells treated with CmIgG-DM1. Co-localization of RON with LAMP1 was not observed in these cells. Thus, results from FIG. 2 demonstrate that Zt/g4-DM1 is effective in induction of RON endocytosis by CRC cells.

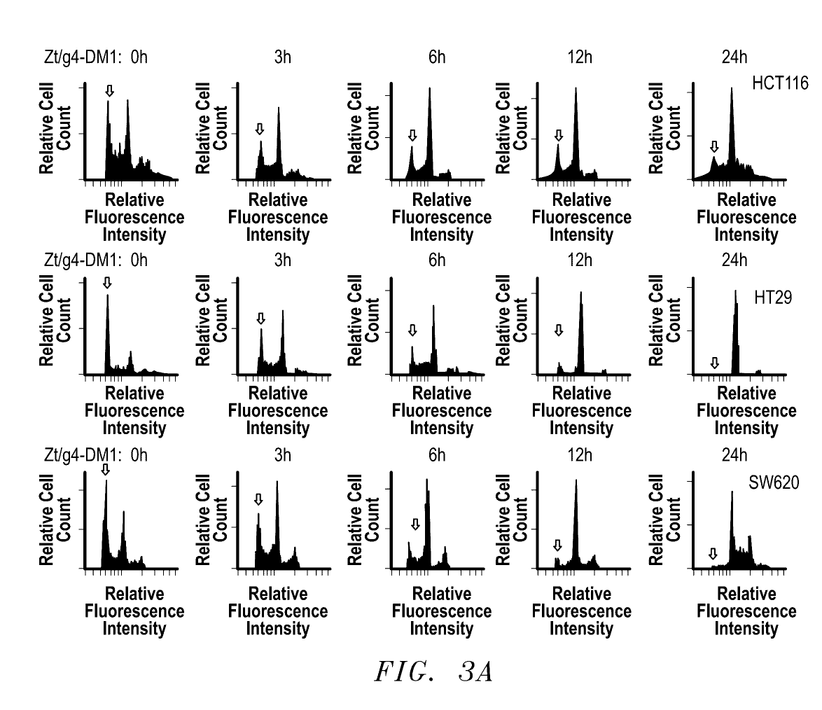

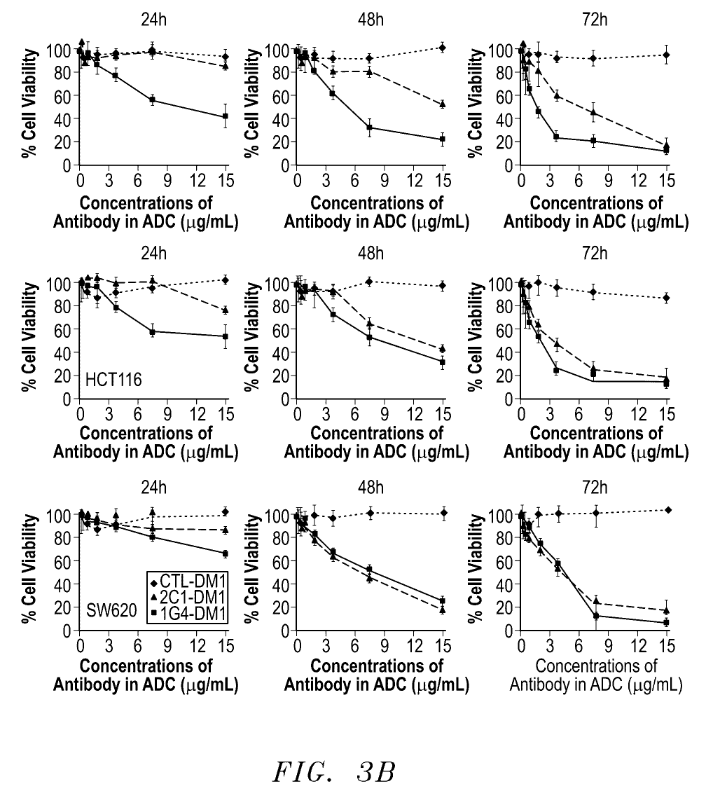

Effect of Zt/g4-DM1 on CRC cell cycle, growth, and death: DM1 acts on microtubules to cause cell cycle arrest at G2/M phase followed by cell death (29, 34, 35). Zt/g4 intracellular delivery of DM1 results in cell cycle changes. The changes in cell cycle profile were observed as early as 3 h after addition of Zt/g4-DM1, featuring a significant reduction in G0/G1 phase, a decrease in S phase, and a dramatic increase in G2/M phase (FIG. 3A). These changes were present in all three CRC cell lines tested. Quantitative measurement of cell cycle changes at 24 h (data not shown). CmIgG-DM1 treatment had minimal effect on cell cycles compared to those from the Zt/g4-DM1 treated cells. Thus, Zt/g4-targeted delivery of DM1 affects cell cycles in CRC cells.

The effect of Zt/g4-DM1 on cell viability was determined. Sensitivity of CRC cells to free DM1 (data not shown) with IC₅₀ values at 4.1 nM for HCT116, 4.4 nM for HT29, and 3.2 nM for SW620 cells, which suggests high sensitivity to DM1. The cells were treated with Zt/g4-DM1. A significant reduction in cell viability was observed in a time and dose-dependent manner (FIG. 3B). The IC₅₀ value of Zt/g4-DM1 at 72 h was 1.64 μg/ml for HT29, 2.16 μg/ml for HCT116, and 4.03 μg/ml for SW620 cells, respectively. The effect of Zt/c1-DM1 was relatively weak with IC₅₀ values at 6.26 μg/ml for HT29, 4.64 μg/ml for HCT116, and 4.36 μg/ml for SW620 cells, respectively. Both Zt/g4-DM1 and Zt/c1-DM1 had no effect on RON-negative LoVo cells. DLD1 cells showed a slight reduction in cell viability with IC₅₀ value at 20.36 μg/ml (data not shown). This shows that anti-RON ADC is ineffective in CRC cells expressing low levels of RON (below 5,000 sites per cell). A comparison of the Zt/g4-DM1 efficacy among four CRC cell lines with the different number of RON receptor per cells (data not shown). Thus, Zt/g4-DM1 is more efficient than Zt/c1-DM1 in reducing viability of CRC cells expressing high levels of RON.

Morphological observation indicated a massive cell death 72 h after cells were exposed to Zt/g4-DM1 (FIG. 3C). More than 50% cell death was observed 72 h after cells were treated with 7.5 mg/ml Zt/g4-DM1 (FIG. 3D). The IC₅₀ value ranged at 5-7 μg/ml in all three CRC cell lines tested. We also counted viable cells 72 h after incubation of 1×10⁴ CRC cells per well in the presence of Zt/g4-DM1. Zt/g4-DM1 treatment results in a significant reduction in the number of viable cells (data not shown). Thus, Zt/g4-DM1 not only causes cell cycle arrest and reduces cell viability, but also reduces viable cell numbers and induces massive CRC cell death.

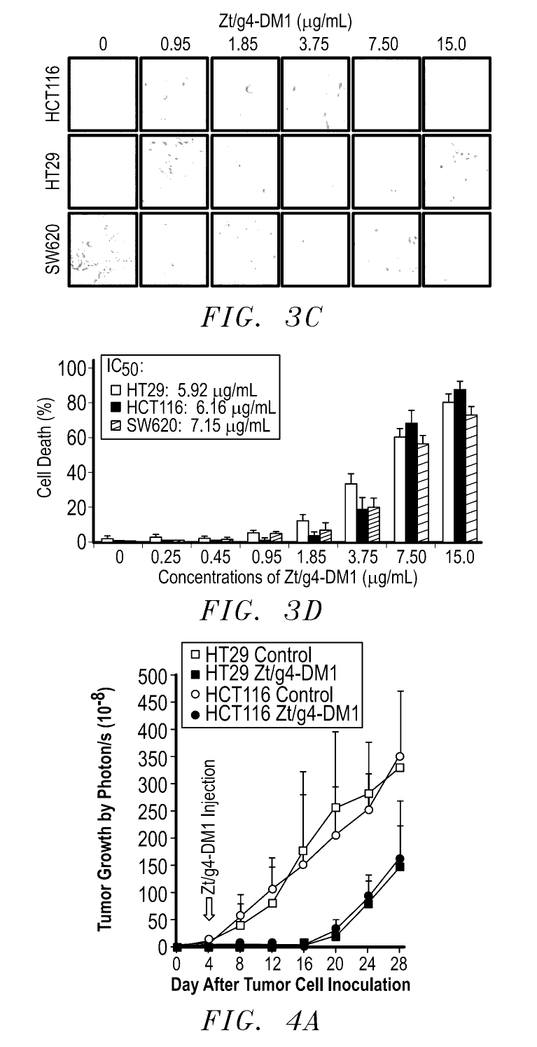

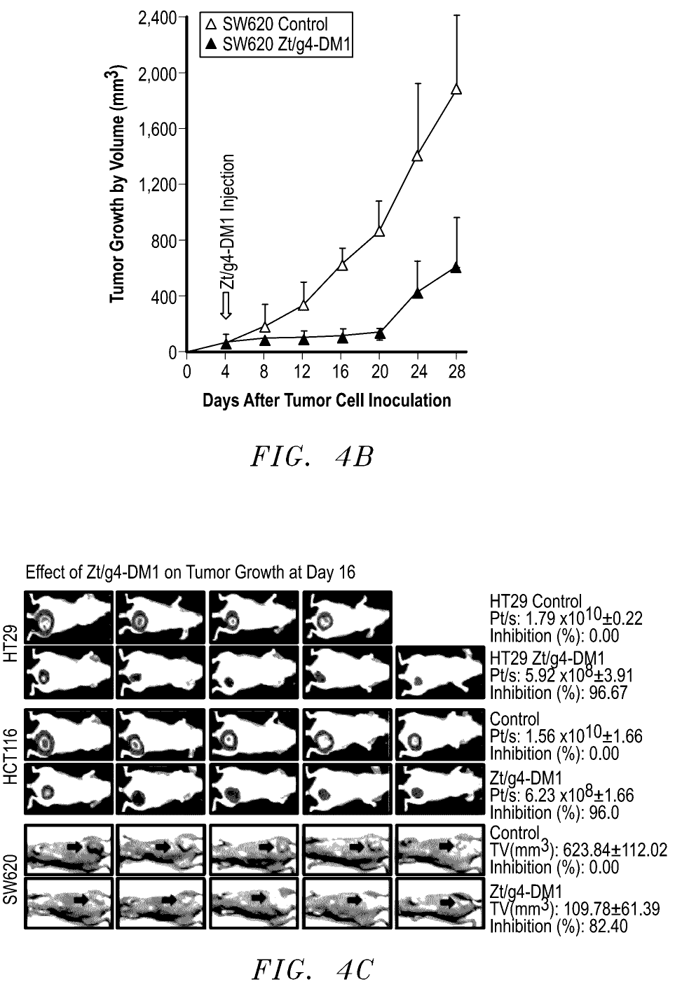

Therapeutic activity of Zt/g4-DM1 in mouse xenograft tumor model. The inventors first determined the efficacy of a single dose of Zt/g4-DM1 at 20 mg/kg body weight on tumors derived from HCT116, HT29, and SW620 cells. Tumor growth by HCT116-luc2 and HT29-luc2 cells was measured by bioluminescence emitted from tumor cells. SW620-mediated tumors were evaluated by tumor volume (18,34). A single dose of Zt/g4-DM1 at 20 mg/kg is sufficient to delay tumor growth caused by all three CRC cell lines (FIGS. 4A and 4B). This time-dependent inhibition was statistically significant. Images of tumors obtained at day 16 are shown in FIG. 4C. More than 95% inhibition, measured by average bioluminescence intensity, was achieved in both HT29 and HCT116 tumor models. Similar results were observed in mice bearing SW620 tumors. In this case, an average 82% inhibition in tumor volume was documented (FIG. 4C). Tumor regrowth was observed at day 20 and thereafter. An accelerated phase was observed from day 24 to 28 (FIGS. 4A and 4B). It is known that mouse IgG1 has a half-life of ˜6 days in vivo (36). Thus, these results show that maintenance of Zt/g4-DM1 at about 5 mg/ml in vivo is required to delay tumor growth (data not shown). Nevertheless, by measuring the average tumor weight at day 28, it was still found that a significant delay in tumor growth was observed in the single dose study. The inhibition rate was 50.98% for HT29, 58.0% for HCT116, and 61.9% for SW620 tumors, respectively (FIG. 4D). Thus, a single dose of 20 mg/kg Zt/g4-DM1 is effective and displays long-lasting activity in inhibition of tumor growth initiated by all three CRC cell lines.

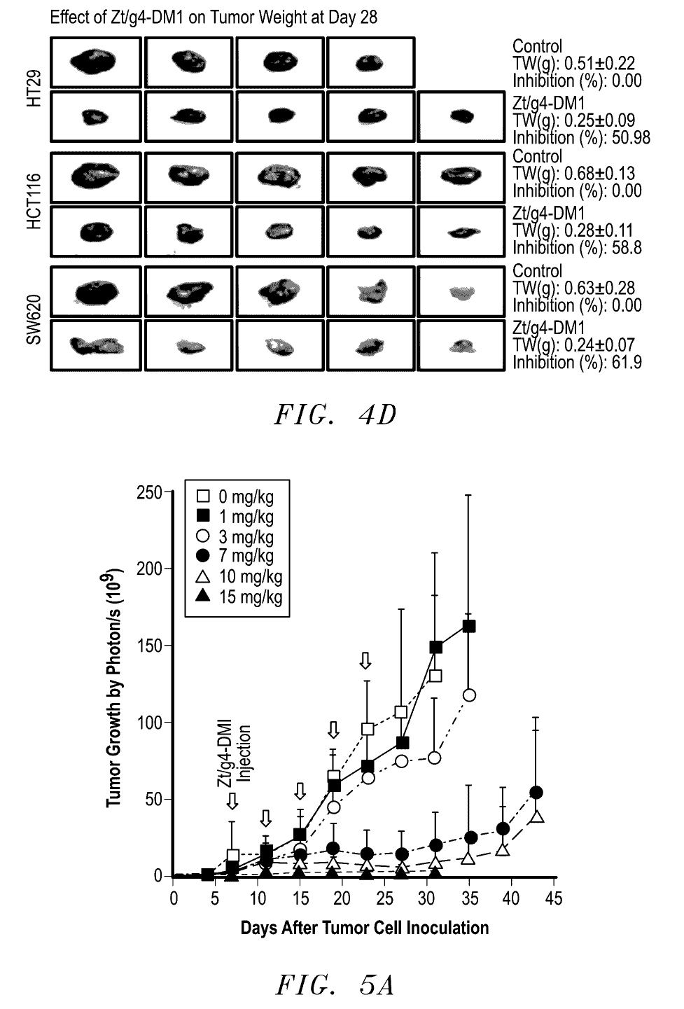

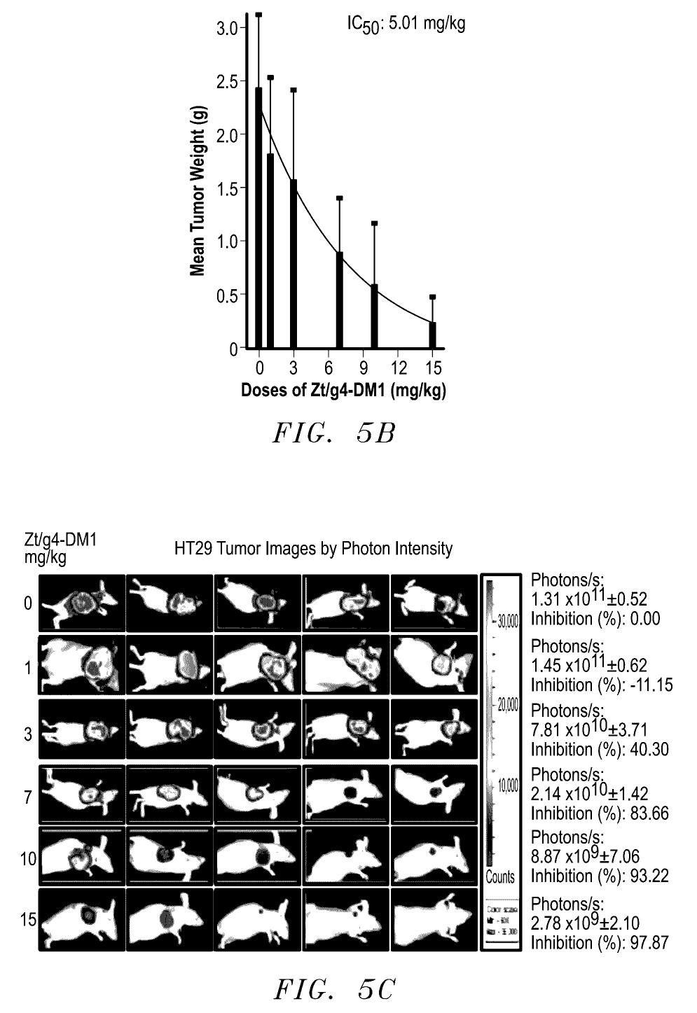

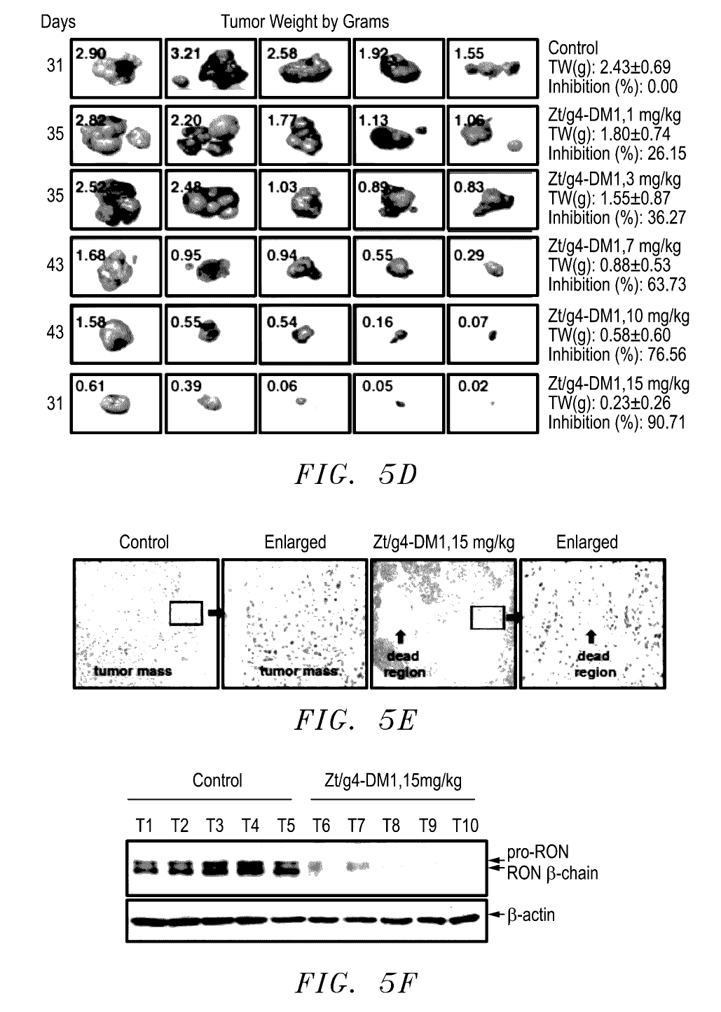

The HT29-Luc2 xenograft tumor model was selected for the dose-ranging study. Mice were injected with different doses of Zt/g4-DM1 once every four days for a total of five injections. Zt/g4-DM1 at 1 or 3 mg/kg showed no inhibition of tumor growth (FIG. 5A). Significant Inhibition was observed in mice treated with 7 mg/kg Zt/g4-DM1 after the third injection. In this case, more than 80% inhibition, calculated by the average photon emission, was obtained from day 19 to 43. The efficacy was more prominent in mice treated with 10 and 15 mg/kg Zt/g4-DM1. In both cases, tumor growth was dramatically delayed after the second injection. Repeated injections at both doses kept tumor growth at minimal levels during the entire period of therapy. By analyzing the average photons at day 31, the IC₅₀ dose for this multi-dose study was 5.01 mg/kg body weight (FIG. 5B). Images of tumors from different groups at day 31 are shown in FIG. 5C. In mice treated with Zt/g4-DM1 at 7, 10 and 15 mg/kg, inhibition was in a dose-dependent manner. More than 95% inhibition in mice treated with 10 and 15 mg/kg Zt/g4-DM1 was achieved compared to that of control mice (FIG. 5C). The average tumor weight from the control mice and the mice treated with 15 mg/kg Zt/g4-DM1 at day 31 was compared to determine the rate of inhibition. A 90% inhibition at average tumor weight was observed (FIG. 5D). Tumors were collected at day 33 (for 1 and 3 mg/kg groups) and day 43 (for 7 and 10 mg/kg groups) and compared with tumors from control group. Significant inhibition was still observed for mice treated with 7 and 10 mg/kg Zt/g4-DM1. Thus, Zt/g4-DM1 at the regimens of 7, 10, 15 mg/kg Q 4 days×5 with a total dose of 35, 50, and 75 mg, respectively, is highly effective in delaying HT29 cell-mediated tumor growth in mouse xenograft models.

To determine if cell death occurs in xenograft tumors, HT29 cell-derived tumor samples collected at day 31 from both control and 15 mg/kg-treated mice were processed for histological analyses. Analysis by H&E staining revealed cell death in different regions in all Zt/g4-DM1-treated tumors but not in control samples (FIG. 5E). An average percentage of dead areas in a tumor mass were 65%±7.4. Western blot analysis using cell lysates from tumor samples also showed that RON expression in Zt/g4-DM1-treated tumors (16.44%±5.75) was dramatically reduced compared to that in control samples (100%±15.56) (FIG. 5F). Thus, Zt/g4-DM1 causes cells death in CRC xenograft tumors, which is associated with elimination of CRC cells overexpressing RON.

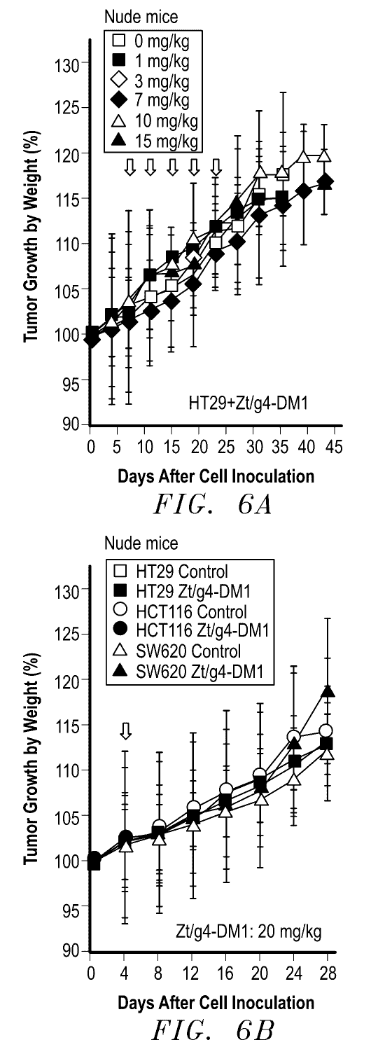

Toxic effect of Zt/g4-DM1 on mice. Three studies using two different types of mice were performed to study Zt/g4-DM1 on animal behavior and body weight. The first study addressed the impact of multi-doses of Zt/g4-DM1. Athymic nude mice were injected five times with 1, 3, 5, 7, 10, 15 mg/kg of Zt/g4-DM1 and monitored every four days for a total period of 31 days. All mice behaved normally during the entire observational period. The average body weight of study groups was comparable to that of control mice with no differences (FIG. 6A). The second study observed the effect of a single dose of Zt/g4-DM1 at 20 mg/kg in nude mice bearing tumors derived from HT29, HCT116, and SW620 cells. No changes in behavior or body weight were observed (FIG. 6B). The third study involved a single-dose injection of Zt/g4-DM1 at 20, 40, and 60 mg/kg in Balb/c mice monitored for 24 days (FIG. 6C). Moderate distress was observed in mice administered with 60 mg/kg Zt/g4-DM1. Also, a moderate reduction of about 6% body weight was observed within the first four days after 60 mg/kg Zt/g4-DM1 injection. Although the average body weight from this group of mice slowly recovered during the observation period, the overall average remained lower than that of control mice with a 19% difference compared to that of control mice at day 24. Thus, Zt/g4-DM1 at the multiple-dose regimen appeared to be well tolerated. However, a single-dose of Zt/g4-DM1 at 60 mg/kg showed a toxic effect on mouse behavior and body weight.

The inventors developed an anti-RON ADC Zt/g4-DM1 for targeted cancer therapy. It is shown herein that Zt/g4-DM1 retains its specificity to RON after conjugation with DM1. The conjugates were stable at 37° C. with minimal dissociation of DM1 from antibody. Binding of Zt/g4-DM1 to CRC cells causes a rapid endocytosis of cell surface RON. Internalized Zt/g4-DM1 results in cell cycle arrest in G2/M phase, followed by cell viability reduction, and massive cell death. Studies from mouse xenograft tumor models confirmed that a single dose of Zt/g4-DM1 at 20 mg/kg is sufficient to inhibit tumor growth with a long-lasting effect up to 20 days. The multiple dose-ranging studies further demonstrated that the therapeutic regimen at 7, 10, 15 mg/kg, every 4 days×5 with a total dose of 35, 50, and 75 mg, respectively, displays strong efficacy in tumor growth inhibition. Furthermore, we showed that Zt/g4-DM1 at doses up to 40 mg/kg has no toxic effect on mouse behavior or body weight. Thus, Zt/g4-DM1 is a novel biotherapeutic with enhanced efficacy for RON-targeted cancer therapy. Humanization of Zt/g4 is described hereinbelow.

Zt/g4 was conjugated to DM1 at appropriate DARs through the thioether linkage (25-27). Consistent with previous reports (26, 27, 29), Zt/g4-DM1 has a favorable conjugation profile. Most conjugates have DARs ranging from 2:1 to 5:1 with the major peak at 4:1. Such a profile is the typical pattern of ADCs using the thioether linkage technology (31). Zt/g4-DM1 is relatively stable. Incubation of Zt/g4-DM1 at 37° C. for 30 days resulted in only 6.5% reduction in DARs of DM1. This data is consistent with previous reports showing that antibodies conjugated with DM1 through thioether linkage are highly stable both in vitro and in vivo under various conditions (31,33). Although the stability of Zt/g4-DM1 under in vivo conditions was not directly determined, it is expected that the conjugates have a similar stability profile due to the similar conjugation method (31,33). The efficacy of in vivo studies using a single dose of Zt/g4-DM1 at 20 mg/kg supports this expectation. In this case, a single injection is sufficient to inhibit tumor growth for almost three weeks, implying that Zt/g4-DM1 is relatively stable in vivo to exert a long-lasting effect. Clearly, the use of thioether linkage provides the practical basis for future development of humanized Zt/g4-DM1.

The selection of Zt/g4 as the leading candidate for DM1 conjugation is based on some of its unique features. Zt/g4 is a mAb highly specific and sensitive to RON, and recognizes an epitope in the RON sema domain (28). The binding of Zt/g4 to RON results in a rapid and efficient RON internalization process. The internalized RON co-localizes with LAMP1, suggesting that the endocytosis could be mediated through a clathrin-dependent pathway (37). Significantly, more than of 80% of cell surface RON is internalized within 48 h after addition of Zt/g4-DM1. In the case of HT29 cells expressing ˜18,800 RON molecules per cell, it translates into 15,000 RON receptors that are internalized within 48 h. This is equivalent to 60,000 DM1 molecules within a single cell, sufficient to cause cell cycle arrest. It is noticed that the kinetics of RON internalization among three CRC cell lines are quite different after addition of Zt/g4-DM1, suggesting the importance of the rate of endocytosis in regulating efficacy of Zt/g4-DM1. Clearly, Zt/g4-induced RON endocytosis facilitates intracellular delivery of DM1 to exert cytotoxic activity. Moreover, Zt/g4 has no agonistic activities in CRC cells expressing RON (18).

The action of DM1 delivered through Zt/g4 was clearly displayed in CRC cells. First, it was shown by flow cytometric analysis that the delivery of DM1 results in cell cycle arrest in G2/M phase, which is a feature of DM1 that impairs microtubule dynamics (35). This effect was observed as early as 3 h after addition of Zt/g4-DM1, which is characterized by progressive reduction of the G1 phase and the accumulation of cells at the G2/M phase. Second, it was observed that targeted delivery of DM1 progressively decreases cell viability. More than 80% reduction in cell viability 72 h after treatment was achieved among the three CRC cell lines tested. Finally, it was documented that a massive cell death in Zt/g4-DM1-treated CRC cells in a dose-dependent manner with IC₅₀ values in the range of 5 to 7 μg/ml Zt/g4-DM1. This evidence demonstrates that DM1 is effectively delivered by Zt/g4 through a targeted pathway, which results in cell cycle arrest, viability reduction, and cell death.

Results from mouse xenograft CRC models prove that Zt/g4-DM1 is highly efficient in inhibition of tumor growth. This conclusion is supported by mouse models using two treatment regimens. The single dose therapy using 20 mg/kg Zt/g4-DM1 was designed to determine if this dose is sufficient to inhibit tumor growth and, if so how long the effect will last. Indeed, Zt/g4-DM1 at 20 mg/kg was highly effective in delaying xenograft tumor growth with a long-lasting effect of almost two weeks. It is known that mouse IgG1 has a half-life of ˜6 days in vivo (36). Administration of 20 mg/kg Zt/g4-DM1 allow monitoring of its efficacy in a four half-life cycle within 24 days. The obtained results confirmed that the efficacy of Zt/g4-DM1 lasts up to 12 days without signs of tumor regrowth (from day 4 to day 16 as shown on FIG. 4A). By calculation, the amount of Zt/g4-DM1 in vivo required to inhibit tumor growth is about 5 mg/kg (data not shown). In other words, a dose of 5 mg/kg Zt/g4-DM1 maintains a balance between tumor growth and inhibition.

The multiple dose-ranging studies were designed to determine the minimum dose required to inhibit xenograft tumor growth. Zt/g4-DM1 at 7 mg/kg in the regimen of Q 4 days×5 with a total dose of 35 mg/kg achieves a significant inhibition. An increase of Zt/g4-DM1 up to 10 and 15 mg/kg in a similar regimen results in a superior therapeutic index. In both cases, the total amount of Zt/g4-DM1 was at 50 and 75 mg/kg, respectively. These results show an IC₅₀ value of 5.01 mg/kg (calculated according to the repeated Zt/g4-DM1 administration and the estimated antibody half-life), which is consistent with the estimated values of 5 mg/kg from the single dose study. Thus, results from multiple dose regimens can be used to determine the optimal treatment regimen for a humanized Zt/g4-DM1.

Analysis of the toxic profile in two types of mice indicates that Zt/g4-DM1 is relatively safe at therapeutic doses with minimal impact on animal's behavior and body weight. Since Zt/g4 does not recognize mouse RON, the observed low toxicity suggest a very limited dissociation of the Zt/g4-DM1 conjugates in vivo. However, a single dose of Zt/g4-DM1 at 60 mg/kg has a negative impact on mouse highlighted by an average of 6% to 19% reduction of body weight during the entire period of study. This suggests that during the administration of multiple doses of Zt/g4-DM1, the accumulated Zt/g4-DM1 in vivo should not exceed the 60 mg/kg limitation. This dose limitation should be a valuable reference for the use of humanized Zt/g4-DM1 in human subjects in the future.

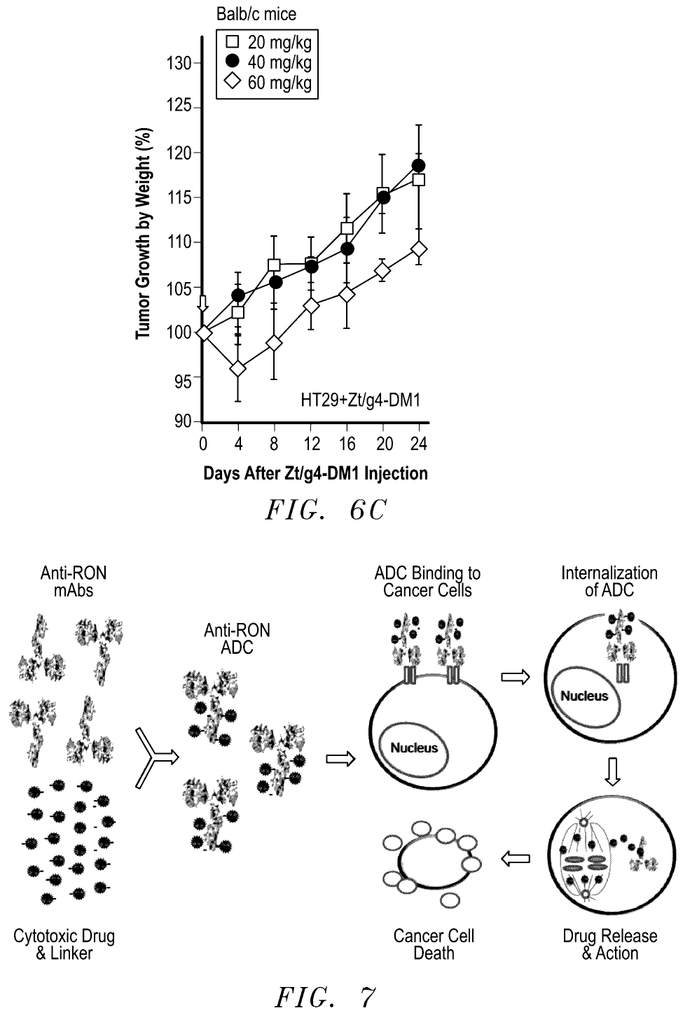

FIG. 7 shows a schematic of the use of the monoclonal antibodies of the present invention in which the various anti-RON monoclonal antibodies of the present invention, which bind RON with high affinity and lead to endocytosis of cancer cells that express RON, in which the anti-RON antibodies carry a cytotoxic drug bound to the anti-RON monoclonal antibody(ies) of the present invention, which can be attached, e.g., covalently, to the anti-RON monoclonal antibody, and which may also include a linker (e.g., a peptide linker, a chemical linker, etc.) to form an antibody drug conjugate (ADC). The ADCs bind the target cells and the antibody portion of the ADC triggers cancer cell internalization of the ADC, the cytotoxic drug is released in the target cell, leading to cancer cell death.

Table 1 summarizes the cytotoxic effect of Zt/g4-DM1 and Zt/c1-DM1 on human colorectal cancer HT-29 cells.

| | IC₅₀ value | IC₅₀ value |
|---|---|---|
| Anti-RON mAb-DM1 | (μg/mL) | (nM) |
| Zt/g4-DM1 | 1.25 | 8.32 |
| Zt/c1-DM1 | 4.43 | 29.51 |
| Zt/c1-DM1-1 | 5.08 | 33.89 |

Mouse Monoclonal Antibody Zt/f2 Specific to Human RON: Zt/F2 binding sequences and amino acids.

Heavy chain: DNA sequence (429 bp) that encompasses the variable region (remainder of the sequence encompassing constant regions, which can be made into fusion proteins using methods and sequences that are well-known in the art, e.g., human constant and framework regions to make humanized antibodies. In the sequence below, the framework regions are in bold, and the complementarity determining regions (CDRs) are underlined for both the nucleic acid and amino acid sequences.

Leader sequence-FR1-CDR1-FR2-CDR2-FR3-CDR3-FR4:
(SEQ ID NO.: 1)
ATGGAAAGGCACTGGATCTTTCTCTTCCTGATTTCAGTAACTGCAGGT
GTCCACTCCCAGGTCCAACTTCAGCAGTCTGGGGCTGAACTGGCAAAA
CCTGGGGCCTCAGTGAAGATGTCCTGCAAGGCGTCTGGCTACACCTTT
ACTAGCTACTGGATGCACTGGGTAAAACAGAGGCCTGGACAGGGTCTG
GAATGGATTGGATACATTAATCCTAGCACTGGTTATATTGAGTACAAT
CAGAACTTCAAGGACAAGGCCACATTGACTGCAGACAAATCCTCCAGC
ACAGCCTACATGCAACTGAGCAGCCTGACATCTGAGGACTCTGCAGTC
TATTACTGTGCAAGATCCCCCTCTCATTATTACGGTAGTAGGTACGGA
TATTTCGATGTCTGGGGCGCAGGGACCACGGTCACCGTCTCCTCA

Heavy chain: Amino acids sequence (143 AA). In the sequence below, the framework regions are in bold, and the complementarity determining regions (CDRs) are underlined for both the nucleic acid and amino acid sequences.

Leader sequence-FR1-CDR1-FR2-CDR2-FR3-CDR3-FR4.
(SEQ ID NO.: 2)
MERHWIFLFLISVTAGVHSQVQLQQSGAELAKPGASVKMSCKASGYTF
TSYWMHWVKQRPGQGLEWIGYINPSTGYIEYNQNFKDKATLTADKSSS
TAYMQLSSLTSEDSAVYYCARSPSHYYGSRYGYFDVWGAGTTVTVSS

Light chain: DNA sequence (384 bp). In the sequence below, the framework regions are in bold, and the complementarity determining regions (CDRs) are underlined for both the nucleic acid and amino acid sequences.

Leader sequence-FR1-CDR1-FR2-CDR2-FR3-CDR3-FR4:
(SEQ ID NO.: 3)
ATGGATTTTCAAGTGCAGATTTTCAGCTTCCTGCTAATCAGTGCTTCA
GTCATAATGTCCAGAGGACAAATTGTTCTCTCCCAGTCTCCAGCAATC
CTGTCTGCATCTCCAGGGGAGAAGGTCACAATGACTTGCAGGGCCAGC
TCAAGTGTAAGTTACATGCACTGGTACCAGCAGAAGCCAGGATCCTCC
CCCAAACCCTGGATTTATGCCACATCCAACCTGGCTTCTGGAGTCCCT
GCTCGCTTCAGTGGCAGTGGGTCTGGGACCTCTTACTCTCTCACAATC
AGCAGAGTGGAGGCTGAAGATGCTGCCACTTATTACTGTCAGCAGTGG
AGTAGTAACCCACGGACGTTCGGTGGAGGCACCAAGCTGGAAATCAAA

Light chain: Amino acids sequence (128 AA). In the sequence below, the framework regions are in bold, and the complementarity determining regions (CDRs) are underlined for both the nucleic acid and amino acid sequences.

Leader sequence-FR1-CDR1-FR2-CDR2-FR3-CDR3-FR4:
(SEQ ID NO.: 4)
MDFQVQIFSFLLISASVIMSRGQIVLSQSPAILSASPGEKVTMTCRAS
SSVSYMHWYQQKPGSSPKPWIYATSNLASGVPARFSGSGSGTSYSLTI
SRVEAEDAATYYCQQWSSNPRTFGGGTKLEIK

Mouse Monoclonal Antibody Zt/g4 Specific to human RON: Zt/g4 binding sequences and amino acids.

Heavy chain: DNA sequence (414 bp). In the sequence below, the framework regions are in bold, and the complementarity determining regions (CDRs) are underlined for both the nucleic acid and amino acid sequences.

Leader sequence-FR1-CDR1-FR2-CDR2-FR3-CDR3-FR4:
(SEQ ID NO.: 5)
ATGAAATGCAGCTGGGTTATCTTCTTCCTGATGGCAGTGGTCACAGGGG
TCAATTCAGAGGTTCAGCTGCAGCAGTCTGGGGCAGAACTTGTGAAGCC
AGGGGCCTCAGTCAAGTTGTCCTGCACAACTTCTGGCTTCAACATTATA
GACACCTATATACACTGGGTGAATCAGAAGCCTGATCAGGGCCTGGAGT
GGATTGGAAGGATTGACCCTGCGGATGGTAATAGAAAATCTGACCCGAA
GTTCCAGGTCAAGGCCACAATAACTGTTGACACATCCTCCAACACAGCC
TACCTGCAACTCAGCAGCCTGACATCTGGGGACACTGCCGTCTATTACT
GTGCCAGAGGGTACGGTAACCTCAATGCTATGGACTCCTGGGGTCAAGG
AACCTCAGTCACCGTCTCCTCA

Heavy chain: Amino acids sequence (138 AA). In the sequence below, the framework regions are in bold, and the complementarity determining regions (CDRs) are underlined for both the nucleic acid and amino acid sequences.

Leader sequence-FR1-CDR1-FR2-CDR2-FR3-CDR3-FR4:
(SEQ ID NO.: 6)
MKCSWVIFFLMAVVTGVNSEVQLQQSGAELVKPGASVKLSCTTSGFNII
DTYIHWVNQKPDQGLEWIGRIDPADGNRKSDPKFQVKATITVDTSSNTA
YLQLSSLTSGDTAVYYCARGYGNLNAMDSWGQGTSVTVSS

Light chain: DNA sequence (381 bp). In the sequence below, the framework regions are in bold, and the complementarity determining regions (CDRs) are underlined for both the nucleic acid and amino acid sequences.

Leader sequence-FR1-CDR1-FR2-CDR2-FR3-CDR3-FR4:
(SEQ ID NO.: 7)
ATGAGGGTCCTTGCTGAGCTCCTGGGGCTGCTGCTGTTCTGCTTTTTAG
GTGTGAGATGTGACATCCAGATGAACCAGTCTCCATCCAGTCTGTCTGC
ATCCCTTGGGGACACAATTACCATCACTTGCCATGCCAGTCAGAACATT
AATGTTTGGTTAAACTGGTATCAGCAGAAACCCGGAAATATTCCTAAAC
TATTGATCTATAAGGCTTCCAACTTGCACACAGGCGTCCCATCAAGGTT
TAGTGGCAGTGGATCTGGAACAGGTTTCACATTAACCATCAGCAGCCTG
CAGCCTGAAGACATTGCCACTTACTACTGTCAACAGGGTCAAAGTTATC
CTCTGACGTTCGGTGGAGGCACCAAGCTGGAAATCAAA

Light chain: Amino acids sequence (127 AA). In the sequence below, the framework regions are in bold, and the complementarity determining regions (CDRs) are underlined for both the nucleic acid and amino acid sequences.

Leader sequence-FR1-CDR1-FR2-CDR2-FR3-CDR3-FR4
(SEQ ID NO.: 8)
MRVLAELLGLLLFCFLGVRCDIQMNQSPSSLSASLGDTITITCHASQNI
NVWLNWYQQKPGNIPKLLIYKASNLHTGVPSRFSGSGSGTGFTLTISSL
QPEDIATYYCQQGQSYPLTFGGGTKLEIK

In one non-limiting example, the following amino acid sequences can be veneered into the CDR regions within the framework sequences of another antibody, e.g., a human antibody backbone, using the following CDRs: heavy chain CDRs selected from: GYTFTSYWMH (SEQ ID NO.:9), YINPSTGYIEYNQNFKD (SEQ ID NO.:10), and SPSHYYGSRYGYFDV (SEQ ID NO.:11); or heavy chain GFNIIDTYIH (SEQ ID NO.:15), RIDPADGNRKSDPKFQV (SEQ ID NO.:16), and GYGNLNAMDS (SEQ ID NO.:17). Likewise, the light chains can also be substituted, with the light chain CDRs selected from: RASSSVSYMH (SEQ ID NO.:12), ATSNLAS (SEQ ID NO.:13), and QQWSSNPRT (SEQ ID NO.:14); or HASQNINVWLN (SEQ ID NO.:18), KASNLHT (SEQ ID NO.:19), and QQGQSYPLT (SEQ ID NO.:20).

Anti-RON Antibody-Drug Conjugates Zt/g4-DM1 in Combination with Chemotherapeutics as a Novel Therapeutic Strategy for Advanced Pancreatic Cancers.

Pancreatic ducal adenocarcinoma (PDAC) is one of the most malignant tumors with limited treatment options. Every effort has been made to develop novel therapeutics to combat this deadly disease. The present inventors further provide a novel biotherapeutic known as anti-RON antibody Zt/g4-drug maytansinoid (DM1) conjugates (anti-RON ADCs) and its combination with chemoagents for targeted treatment of advanced PDAC. Zt/g4 is a mouse monoclonal antibody (IgG1α/κ) highly specific to human RON. Conjugation of Zt/g4 to DM1 to form Zt/g4-DM1 was achieved using thioether linkage technique. Zt/g4 was also conjugated to monomethyl auristatin E (MMAE) to form Zt/g4-MMAE. The generated anti-RON ADCs have an average drug to antibody ratio of 3.8:1.

Using human PDAC cell lines L3.6p1, BxPC-3, and FG as the model, we found that both Zt/g4-DM1 and Zt/g4-MMAE are highly efficient in induction of RON endocytosis, which leads to specific delivery of cytotoxic payloads to cancer cells. The targeted delivery resulted in cell cycle arrest at G2/M phases, reduced cell viability, and massive cell death. Among three PDAC cell lines tested, the average IC50 values for Zt/g4-DM1 and Zt/g4-MMAE in causing cell death were 3.13 μg/ml and 5.16 μg/ml, respectively. Anti-RON ADCs also showed a synergism in vitro with chemotherapeutics including gemcitabine to kill PDAC cells. In mouse PDAC xenograft models, Zt/g4-DM1 was highly effective in inhibiting PDAC cell-mediated tumor growth in a time-dose fractionation study. In vivo studies of Zt/g4-DM1 in combination with gemcitabine are currently underway. The present inventors demonstrate herein that anti-RON ADCs Zt/g4-DM1 or Zt/g4-MMAE are novel biotherapeutics highly specific to PDAC cells expressing RON. Confirmation of anti-RON ADCs' effectiveness in preclinical PDAC models demonstrates the efficacy of humanized anti-RON ADCs.

Cell Lines and Reagents: Panc-1, L3.6PL, and BxPC-3 cell lines were from ATCC. FG cells were from Dr. A M. Lowy (Moores Cancer Center UC San Diego). Mouse anti-RON mAbs Zt/g4 and Zt/c1 were produced as disclosed hereinabove.

Conjugation of anti-RON mAb with DM1: Zt/g4 was conjugated to SMCC-DM1 to achieve a drug-antibody ratio (DAR) of 4:1 via thioether linkage to form Zt/g4-DM1. Conjugates were purified using a PC10 column, sterilized through a 0.22 μM filter, and stored at 4° C. Analysis of Zt/g4-DM1 was determined by hydrophobic interaction chromatography using a Varian Prostar 210 Quaternary HPLC system (Varian, Palo Alto, Calif., USA) coupled with a TSK butyl-NPR 4.6×3.5 column (Tosoh Biosciences, Prussia, Pa.).

Immunofluorescence analyses: Immunofluorescence detection of cell surface or cytoplasmic RON was performed by incubating cells with 5 μg/ml Zt/g4 or Zt/g4-DM1 followed by goat anti-mouse IgG coupled with FITC or rhodamine. Normal mouse IgG was used as the control.

In vitro cell viability and death assays: Cell viability after treatment of Zt/g4-DM1, chemotherapeutics, and their combinations was determined by the MTT assay. Cell death was determined by the trypan blue exclusion assay.

Flow cytometric analysis of cell cycle: Cell cycle was determined by incubating cells with Zt/g4-DM1, labeled with propidium iodide, and then analyzed by an Accuri Flow Cytometer.

Mouse xenograft PDAC model and anti-RON ADC treatment: Female athymic nude mice were injected with 5×106 L3.6PL, BxPC-3, or FG cells into the subcutaneous space of the right flank. Treatment began when all tumors have reached an average tumor volume of 100 to 200 mm3 The single-dose effect was studied by injection of 20 mg/kg Zt/g4-DM1. Tumor volumes were determined every four days using a previously described formula: V=pi/6×1.58×(length×width)3/2.

Statistical analysis: GraphPad Prism 6 software was used for statistical analysis. Results are shown as mean±SD. The data between control and experimental groups were compared using Student t test. Statistical differences at p<0.05 were considered significant.

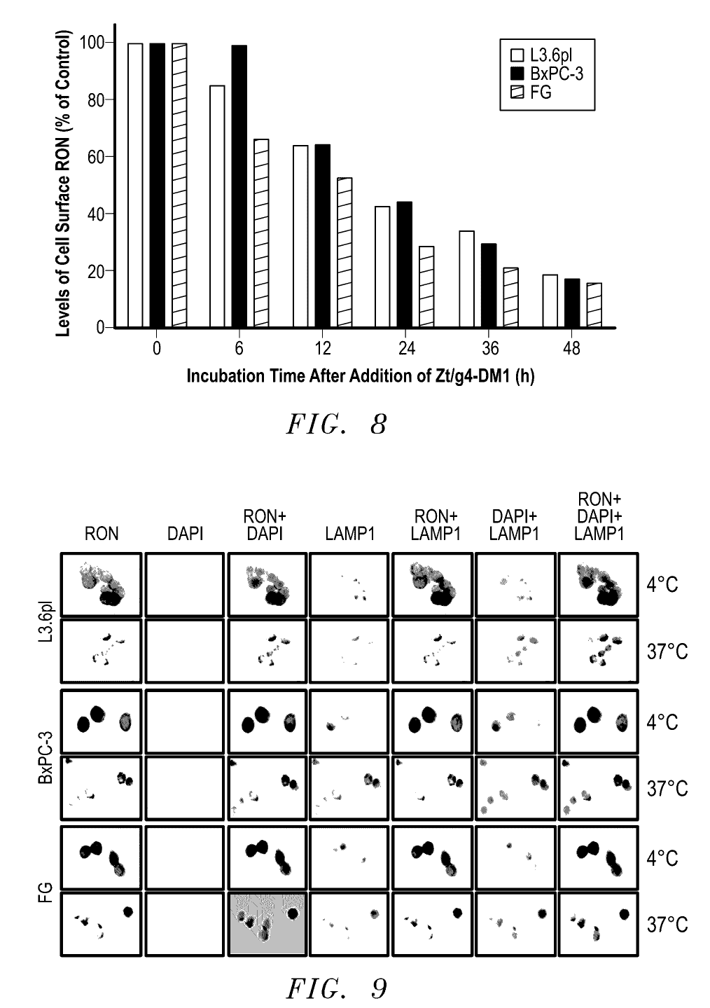

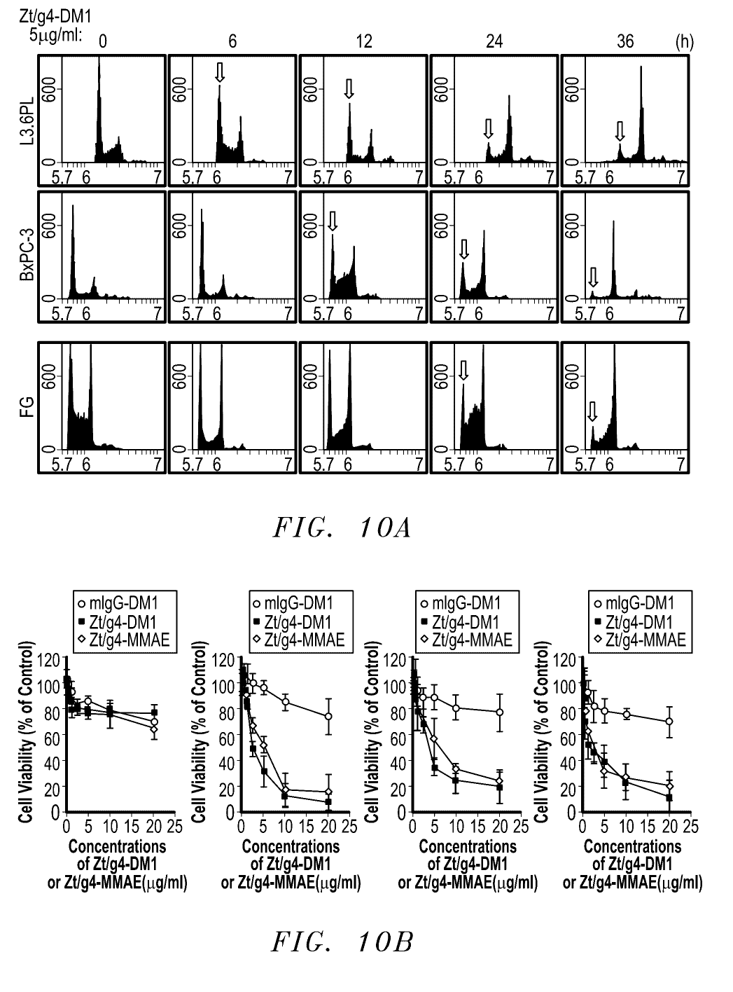

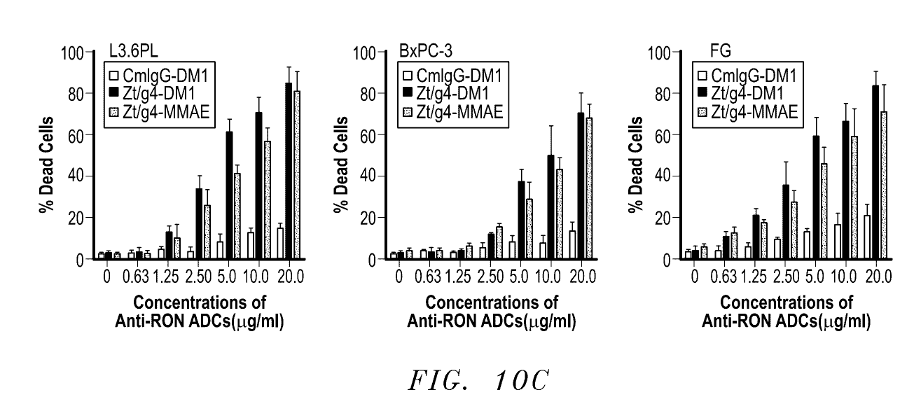

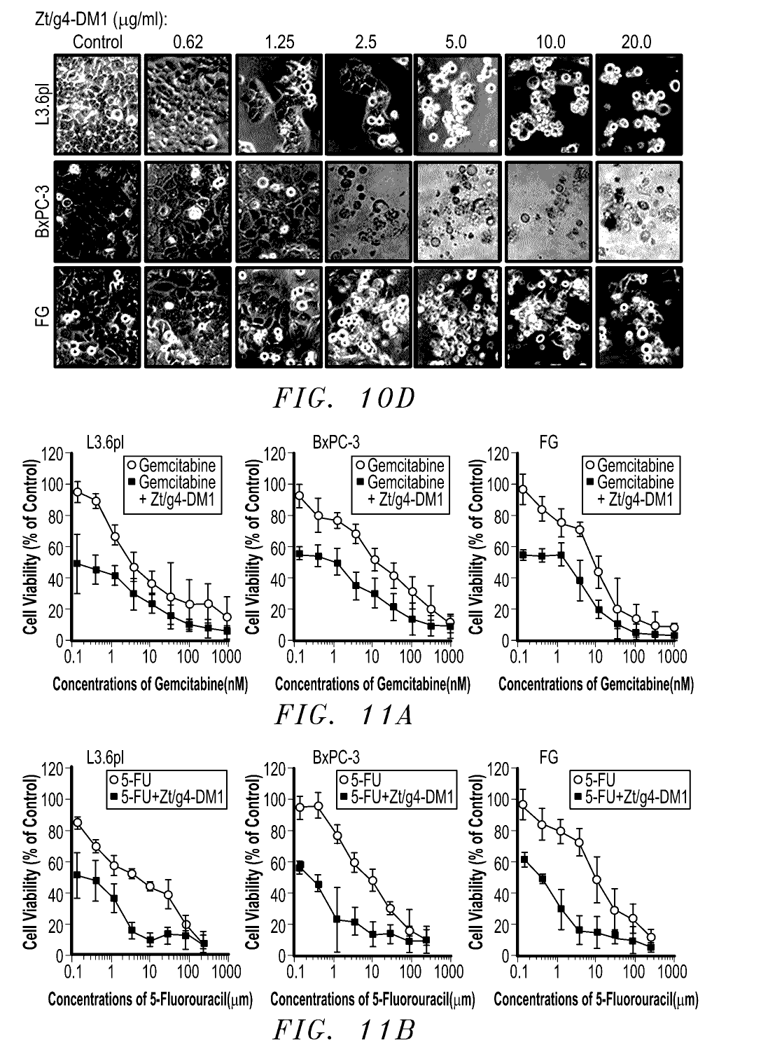

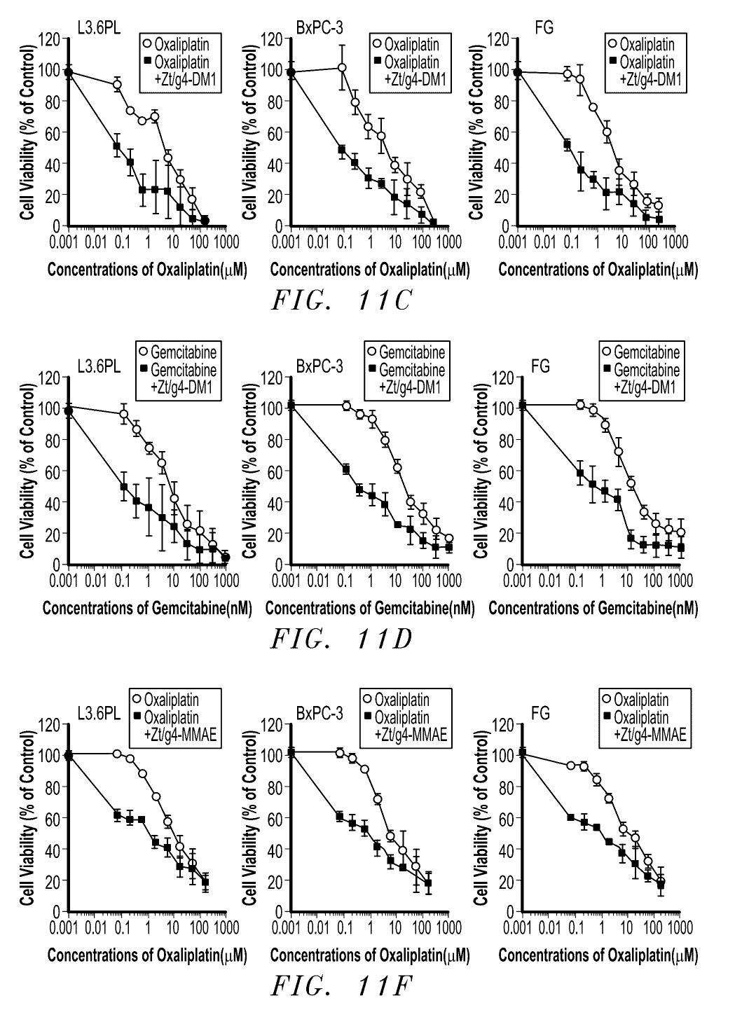

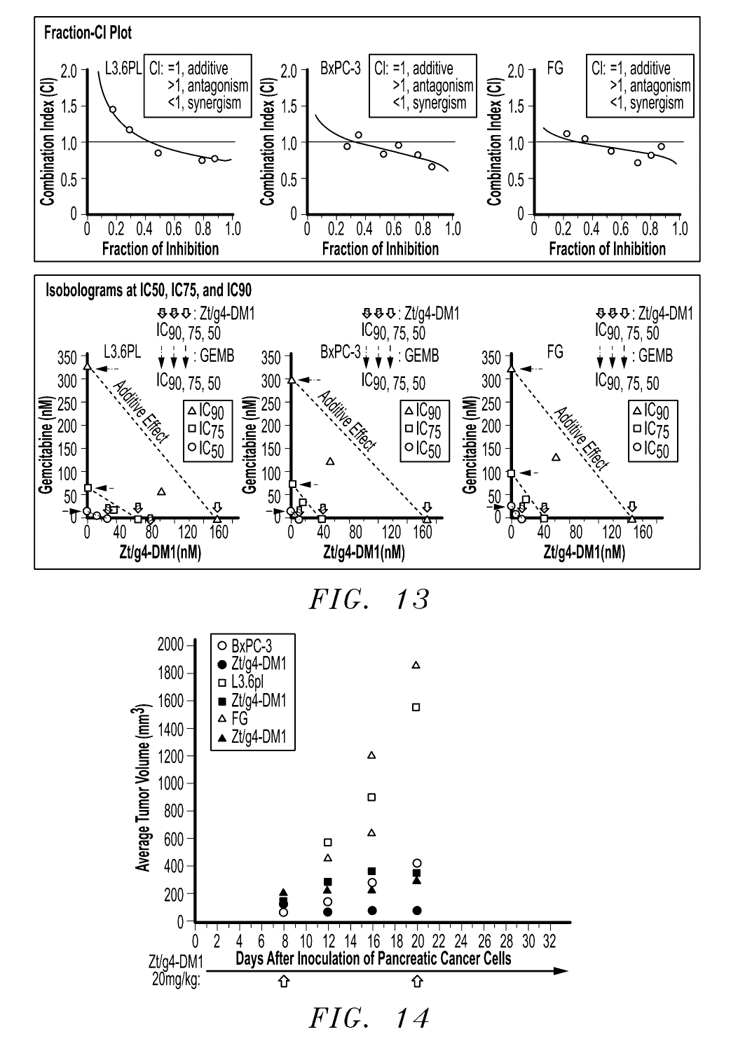

FIG. 8 is a graph that shows that Zt/g4-DM1 induces cell surface RON reduction in pancreatic cancer cell lines. FIG. 9 shows the Zt/g4-DM1-induced intracellular RON localization in pancreatic cancer cells. FIGS. 10A to 10D are graphs that show the effect of Zt/g4-DM1 on pancreatic cancer cell cycle, viability, and apoptotic death. FIGS. 11A to 11C are graphs that show a synergistic activity of Zt/g4-DM1 in combination with different chemotherapeutics. FIG. 11D includes graphs that show a synergistic activity of Zt/g4-MMAE in combination with Gemcitabine and viability of human pancreatic cancer cells; and FIG. 11E shows graphs that show the synergistic activity of Zt/g4-MMAE in combination with Oxaliplatin and viability of human pancreatic cancer cells. FIG. 12 are graphs that show synergism between Zt/g4-DM1 and chemotherapeutics by isobolograms. FIG. 13 is a graph that shows the therapeutic effect of Zt/g4-DM1 at a single dose on xenograft growth of human PDACs.

Thus, it is demonstrated herein that Zt/g4-DM1 is highly effective in inhibition of xenograft PDAC growth in vivo in a human xenograft mouse model. Zt/g4-DM1 in combination with chemotherapeutics shows synergistic effect on PDAC cell viability.

Sequences from mouse anti-RON mAb Zt/g4 CDR and framework regions in both heavy and light chains were grafted into human IgG1 acceptor framework to create five humanized light (L1-5) chains and five humanized heavy (H1-5) chains as shown below. This results in twenty-five different parings of humanized Zt/g4. Among them, humanized Zt/g4 H1L2, H1L3, and H3L2 have been used for antibody-drug conjugation.

The sequences are shown in the following format: Kozak sequence followed by a Leader sequence shown in italics, the variable region (VH/VL) shown in BOLD, the constant region (hIgG1CH/hIgKCL) shown in underline, and the final three nucleic acids are stop codons.

### DNA Sequences

>G4-hzVH1-hIgG1CH
(SEQ ID NO: 21)
GCCGCCACCATGGGCTGGAGCTGGATCCTGCTGTTCCTCCTGAGCGTGACAGCAGGAGTGCACAGCCAGGTGC
AGCTGGTGCAGTCTGGGGCTGAGGTGAAGAAGCCTGGGGCTACAGTGAAAATCTCCTGCAAGGTTTCTGGATA
CACCTTCACCGACACCTATATACACTGGGTGCAACAGGCCCCTGGAAAAGGGCTTGAGTGGATGGGAAGGATT
GACCCTGCGGATGGTAATAGAAAATCTGACCCGAAGTTCCAGGTCAGAGTCACCATAACCGCGGACACGTCTA
CAGACACAGCCTACATGGAGCTGAGCAGCCTGAGATCTGAGGACACGGCCGTGTATTACTGTGCAAGAGGGTA
CGGTAACCTCAATGCTATGGACTCCTGGGGCCAAGGTACCCTGGTCACCGTGTCGAGAGCTAGCACCAAGGGC
CCATCGGTCTTCCCCCTGGCACCCTCCTCCAAGAGCACCTCTGGGGGCACAGCGGCCCTGGGCTGCCTGGTCA
AGGACTACTTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCCAAATCTT
GTGACAAAACTCACACATGCCCACCGTGCCCAGCACCTGAACTCCTGGGGGGACCGTCAGTCTTCCTCTTCCC
CCCAAAACCCAAGGACACCCTCATGATCTCCCGGACCCCTGAGGTCACATGCGTGGTGGTGGACGTGAGCCAC
GAAGACCCTGAGGTCAAGTTCAACTGGTACGTGGACGGCGTGGAGGTGCATAATGCCAAGACAAAGCCGCGGG
AGGAGCAGTACAACAGCACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAGCCAAAGGG
CAGCCCCGAGAACCACAGGTGTACACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTACAGCAAGCTCACCGTGGACAAG
AGCAGGTGGCAGCAGGGGAACGTCTTCTCATGCTCCGTGATGCATGAGGCTCTGCACAACCACTACACGCAGA
AGAGCCTCTCCCTGTCTCCGGGTAAATGA
>Zt/g4-hzVH1-hIgG1CH-amino acid sequence
(SEQ ID NO: 22)
AATMGWSWILLFLLSVTAGVHSQVQLVQSGAEVKKPGATVKISCICVSGYTFTDTYIHWVQQAPGKGLEWMGR
IDPADGNRKSDPKFQVRVTITADTSTDTAYMELSSLRSEDTAVYYCARGYGNLNAMDSWGQGTLVTVSRASTK
GPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK*
>G4-hzVH2-hIgG1CH
(SEQ ID NO: 23)
GCCGCCACCATGGGCTGGAGCTGGATCCTGCTGTTCCTCCTGAGCGTGACAGCAGGAGTGCACAGCCAGGTGC
AGCTGGTGCAGTCTGGGGCTGAGGTGAAGAAGCCTGGGGCCTCAGTGAAGGTTTCCTGCAAGGCATCTGGATA
CACCTTCACCGACACCTATATACACTGGGTGCGACAGGCCCCTGGACAAGGGCTTGAGTGGATGGGAAGGATT
GACCCTGCGGATGGTAATAGAAAATCTGACCCGAAGTTCCAGGTCAGAGTCACCATGACCAGGGACACGTCCA
CGAGCACAGTCTACATGGAGCTGAGCAGCCTGAGATCTGAGGACACGGCCGTGTATTACTGTGCGAGAGGGTA
CGGTAACCTCAATGCTATGGACTCCTGGGGCCAGGGAACCCTGGTCACCGTCTCCTCAGCTAGCACCAAGGGC
CCATCGGTCTTCCCCCTGGCACCCTCCTCCAAGAGCACCTCTGGGGGCACAGCGGCCCTGGGCTGCCTGGTCA
AGGACTACTTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCCAAATCTT
GTGACAAAACTCACACATGCCCACCGTGCCCAGCACCTGAACTCCTGGGGGGACCGTCAGTCTTCCTCTTCCC
CCCAAAACCCAAGGACACCCTCATGATCTCCCGGACCCCTGAGGTCACATGCGTGGTGGTGGACGTGAGCCAC
GAAGACCCTGAGGTCAAGTTCAACTGGTACGTGGACGGCGTGGAGGTGCATAATGCCAAGACAAAGCCGCGGG
AGGAGCAGTACAACAGCACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAGCCAAAGGG
CAGCCCCGAGAACCACAGGTGTACACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTACAGCAAGCTCACCGTGGACAAG
AGCAGGTGGCAGCAGGGGAACGTCTTCTCATGCTCCGTGATGCATGAGGCTCTGCACAACCACTACACGCAGA
AGAGCCTCTCCCTGTCTCCGGGTAAATGA
>Zt/g4-hzVH2-hIgG1CH-amino acid sequence
(SEQ ID NO: 24)
AATMGWSWILLFLLSVTAGVHSQVQLVQSGAEVKKPGASVKVSCKASGYTFTDYIHWVRQAPGQGLEWMGRID
PADGNRKSDPKFQVRVTMTRDTSTSTVYMELSSLRSEDTAVYYCARGYGNLNAMDSWGQGTLVTVSSASTKGP
SVFPLAPSSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPGK*
>G4-hzVH3-hIgG1CH
(SEQ ID NO: 25)
GCCGCCACCATGGGCTGGAGCTGGATCCTGCTGTTCCTCCTGAGCGTGACAGCAGGAGTGCACAGCCAGGTCC
AGCTTGTGCAGTCTGGGGCTGAGGTGAAGAAGCCTGGGGCCTCAGTGAAGGTTTCCTGCAAGGCTTCTGGATA
CACCTTCACTGACACCTATATACACTGGGTGCGCCAGGCCCCCGGACAAAGGCTTGAGTGGATGGGAAGGATT
GACCCTGCGGATGGTAATAGAAAATCTGACCCGAAGTTCCAGGTCAGAGTCACCATTACCAGGGACACATCCG
CGAGCACAGCCTACATGGAGCTGAGCAGCCTGAGATCTGAAGACACGGCTGTGTATTACTGTGCGAGAGGGTA
CGGTAACCTCAATGCTATGGACTCCTGGGGCCAGGGAACCCTGGTCACCGTCTCCTCAGCTAGCACCAAGGGC
CCATCGGTCTTCCCCCTGGCACCCTCCTCCAAGAGCACCTCTGGGGGCACAGCGGCCCTGGGCTGCCTGGTCA
AGGACTACTTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCCAAATCTT
GTGACAAAACTCACACATGCCCACCGTGCCCAGCACCTGAACTCCTGGGGGGACCGTCAGTCTTCCTCTTCCC
CCCAAAACCCAAGGACACCCTCATGATCTCCCGGACCCCTGAGGTCACATGCGTGGTGGTGGACGTGAGCCAC
GAAGACCCTGAGGTCAAGTTCAACTGGTACGTGGACGGCGTGGAGGTGCATAATGCCAAGACAAAGCCGCGGG
AGGAGCAGTACAACAGCACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAGCCAAAGGG
CAGCCCCGAGAACCACAGGTGTACACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTACAGCAAGCTCACCGTGGACAAG
AGCAGGTGGCAGCAGGGGAACGTCTTCTCATGCTCCGTGATGCATGAGGCTCTGCACAACCACTACACGCAGA
AGAGCCTCTCCCTGTCTCCGGGTAAATGA
>Zt/g4-hzVH3-hIgG1CH-amino acid sequence
(SEQ ID NO: 26)
AATMGWSWILLFLLSVTAGVHSQVQLVQSGAEVKKPGASVKVSCKASGYTFTDTYIHWVRQAPGQRLEWMGRI
DPADGNRKSDPKFQVRVTITRDTSASTAYMELSSLRSEDTAVYYCARGYGNLNAMDSWGQGTLVTVSSASTKG
PSVFPLAPSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK*
>G4-hzVH4-hIgG1CH
(SEQ ID NO: 27)
GCCGCCACCATGGGCTGGAGCTGGATCCTGCTGTTCCTCCTGAGCGTGACAGCAGGAGTGCACAGCCAGGTCC
AGCTGGTACAGTCTGGGGCTGAGGTGAAGAAGCCTGGGGCCTCAGTGAAGGTCTCCTGCAAGGTTTCCGGATA
CACCCTCACTGACACCTATATACACTGGGTGCGACAGGCTCCTGGAAAAGGGCTTGAGTGGATGGGAAGGATT
GACCCTGCGGATGGTAATAGAAAATCTGACCCGAAGTTCCAGGTCAGAGTCACCATGACCGAGGACACATCTA
CAGACACAGCCTACATGGAGCTGAGCAGCCTGAGATCTGAGGACACGGCCGTGTATTACTGTGCAACAGGGTA
CGGTAACCTCAATGCTATGGACTCCTGGGGCCAAGGGACAATGGTCACCGTCTCTTCAGCTAGCACCAAGGGC
CCATCGGTCTTCCCCCTGGCACCCTCCTCCAAGAGCACCTCTGGGGGCACAGCGGCCCTGGGCTGCCTGGTCA
AGGACTACTTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCCAAATCTT
GTGACAAAACTCACACATGCCCACCGTGCCCAGCACCTGAACTCCTGGGGGGACCGTCAGTCTTCCTCTTCCC
CCCAAAACCCAAGGACACCCTCATGATCTCCCGGACCCCTGAGGTCACATGCGTGGTGGTGGACGTGAGCCAC
GAAGACCCTGAGGTCAAGTTCAACTGGTACGTGGACGGCGTGGAGGTGCATAATGCCAAGACAAAGCCGCGGG
AGGAGCAGTACAACAGCACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAGCCAAAGGG
CAGCCCCGAGAACCACAGGTGTACACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTACAGCAAGCTCACCGTGGACAAG
AGCAGGTGGCAGCAGGGGAACGTCTTCTCATGCTCCGTGATGCATGAGGCTCTGCACAACCACTACACGCAGA
AGAGCCTCTCCCTGTCTCCGGGTAAATGA
>Zt/g4-hzVH4-hIgG1CH-amino acid sequence
(SEQ ID NO: 28)
AATMGWSWILLFLLSVTAGVHSQVQLVQSGAEVKKPGASVKVSCKVSGYTLTDTYIHWVRQAPGKGLEWMGRI
DPADGNRKSDPKFQVRVTMTEDTSTDTAYMELSSLRSEDTAVYYCATGYGNLNAMDSWGQGTMVTVSSASTKG
PSVFPLAPSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK*
>G4-hzVH5-hIgG1CH
(SEQ ID NO: 29)
GCCGCCACCATGGGCTGGAGCTGGATCCTGCTGTTCCTCCTGAGCGTGACAGCAGGAGTGCACAGCCAGGTGC
AGCTGCAGGAGTCGGGCCCAGGACTGGTGAAGCCTTCGGAGACCCTGTCCCTCACCTGCACTGTCTCTGGTGG
CTCCATCAGTGACACCTATATACACTGGATCCGGCAGCCCCCAGGGAAGGGACTGGAGTGGATTGGGAGGATT
GACCCTGCGGATGGTAATAGAAAATCTGACCCGAAGTTCCAGGTCCGAGTCACCATATCAGTAGACACGTCCA
AGAACCAGTTCTCCCTGAAGCTGAGCTCTGTGACCGCTGCGGACACGGCCGTGTATTACTGTGCGAGAGGGTA
CGGTAACCTCAATGCTATGGACTCCTGGGGCCAAGGGACAATGGTCACCGTCTCTTCAGCTAGCACCAAGGGC
CCATCGGTCTTCCCCCTGGCACCCTCCTCCAAGAGCACCTCTGGGGGCACAGCGGCCCTGGGCTGCCTGGTCA
AGGACTACTTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCCAAATCTT
GTGACAAAACTCACACATGCCCACCGTGCCCAGCACCTGAACTCCTGGGGGGACCGTCAGTCTTCCTCTTCCC
CCCAAAACCCAAGGACACCCTCATGATCTCCCGGACCCCTGAGGTCACATGCGTGGTGGTGGACGTGAGCCAC
GAAGACCCTGAGGTCAAGTTCAACTGGTACGTGGACGGCGTGGAGGTGCATAATGCCAAGACAAAGCCGCGGG
AGGAGCAGTACAACAGCACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAGCCAAAGGG
CAGCCCCGAGAACCACAGGTGTACACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTACAGCAAGCTCACCGTGGACAAG
AGCAGGTGGCAGCAGGGGAACGTCTTCTCATGCTCCGTGATGCATGAGGCTCTGCACAACCACTACACGCAGA
AGAGCCTCTCCCTGTCTCCGGGTAAATGA
>Zt/g4-hzVH5-hIgG1CH-amino acid sequence
(SEQ ID NO: 30)
AATMGWSWILLFLLSVTAGVHSQVQLQESGPGLVKPSETLSLTCTVSGGSISDTYIHWIRQPPGKGLEWIGRI
DPADGNRKSDPKFQVRVTISVDTSKNQFSLKLSSVTAADTAVYYCARGYGNLNAMDSWGQGTMVTVSSASTKG
PSVFPLAPSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTISKAKG
QPREPQVYTLPPSDELTKNQVSLTCLVKGFYPSDIAVEWESNGQPENNYKTTPPVLDSDGSFFLYSKLTVDKS
RWQQGNVFSCSVMHEALHNHYTQKSLSLSPGK*
>G4-hzVL1-hIgKCL
(SEQ ID NO: 31)
GCCGCCACCATGGGCTGGAGCTGGATCCTGCTGTTCCTCCTGAGCGTGACAGCAGGAGTGCACAGCGACATCC
AGATGACCCAGTCTCCATCCTCCCTGTCTGCATCTGTAGGAGACAGAGTCACCATCACTTGCCATGCCAGTCA
GAACATTAATGTTTGGTTAAACTGGTATCAGCAGAAACCAGGGAAAGCCCCTAAGCTCCTGATCTACAAGGCT
TCCAACTTGCACACAGGGGTCCCATCAAGGTTCAGTGGAAGTGGATCTGGGACAGATTTTACTTTCACCATCA
GCAGCCTGCAGCCTGAAGATATTGCAACATATTACTGTCAACAGGGTCAAAGTTATCCTCTGACGTTCGGCGG
AGGGACCAAGCTGGAGATCAAACGAACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGTTAG
>Zt/g4-hzVL1-hIgKCL-amino acid sequence
(SEQ ID NO: 32)
AATMGWSWILLFLLSVTAGVHSDIQMTQSPSSLSASVGDRVTITCHASQNINVWLNWYQQKPGKAPKLLIYKA
SNLHTGVPSRFSGSGSGTDFTFTISSLQPEDIATYYCQQGQSYPLTFGGGTKLEIKRTVAAPSVFIFPPSDEQ
LKSGTASVVCLLNNFYPREAKVQWKVDNALQSGNSQESVTEQDSKDSTYSLSSTLTLSKADYEKHKVYACEVT
HQGLSSPVTKSFNRGEC*
>G4-hzVL2-hIgKCL
(SEQ ID NO: 33)
GCCGCCACCATGGGCTGGAGCTGGATCCTGCTGTTCCTCCTGAGCGTGACAGCAGGAGTGCACAGCGACATCC
AGATGACCCAGTCTCCATCCTCCCTGTCTGCATCTGTAGGAGACAGAGTCACCATCACTTGCCATGCCAGTCA
GAACATTAATGTTTGGTTAAACTGGTATCAGCAGAAACCAGGGAAAGCCCCTAAGCTCCTGATCTATAAGGCT
TCCAACTTGCACACAGGGGTCCCATCAAGGTTCAGTGGCAGTGGATCTGGGACAGATTTCACTCTCACCATCA
GCAGTCTGCAACCTGAAGATTTTGCAACTTACTACTGTCAACAGGGTCAAAGTTATCCTCTGACGTTCGGCGG
AGGGACCAAGCTGGAGATCAAACGAACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGTTAG
>Zt/g4-hzVL2-hIgKCL-amino acid sequence
(SEQ ID NO: 34)
AATMGWSWILLFLLSVTAGVHSDIQMTQSPSSLSASVGDRVTITCHASQNINVWLNWYQQKPGKAPKLLIYKA
SNLHTGVPSRFSGSGSGTDFTLTISSLQPEDFATYYCQQGQSYPLTFGGGTKLEIKRTVAAPSVFIFPPSDEQ
LKSGTASVVCLLNNFYPREAKVQWKVDNALQSGNSQESVTEQDSKDSTYSLSSTLTLSKADYEKHKVYACEVT
HQGLSSPVTKSFNRGEC*
>G4-hzVL3-hIgKCL
(SEQ ID NO: 35)
GCCGCCACCATGGGCTGGAGCTGGATCCTGCTGTTCCTCCTGAGCGTGACAGCAGGAGTGCACAGCGACATCC
AGATGACCCAGTCTCCATCCTCCCTGTCTGCATCTGTAGGAGACAGAGTCACCATCACTTGCCATGCCAGTCA
GAACATTAATGTTTGGTTAAACTGGTATCAGCAGAAACCAGGGAAAGTTCCTAAGCTCCTGATCTATAAGGCT
TCCAACTTGCACACAGGGGTCCCATCTCGGTTCAGTGGCAGTGGATCTGGGACAGATTTCACTCTCACCATCA
GCAGCCTGCAGCCTGAAGATGTTGCAACTTATTACTGTCAACAGGGTCAAAGTTATCCTCTGACGTTCGGCGG
AGGGACCAAGGTGGAGATCAAACGAACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGTTAG
>Zt/g4-hzVL3-hIgKCL-amino acid sequence
(SEQ ID NO: 36)
AATMGWSWILLFLLSVTAGVHSDIQMTQSPSSLSASVGDRVTITCHASQNINVWLNWYQQKPGKVPKLLIYKA
SNLHTGVPSRFSGSGSGTDFTLTISSLQPEDVATYYCQQGQSYPLTFGGGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVT
HQGLSSPVTKSFNRGEC*
>G4-hzVL4-hIgKCL
(SEQ ID NO: 37)
GCCGCCACCATGGGCTGGAGCTGGATCCTGCTGTTCCTCCTGAGCGTGACAGCAGGAGTGCACAGCGACATCC
AGGTGACCCAGTCTCCATCCTTCCTGTCTGCATCTGTAGGAGACAGAGTCACCATCACTTGCCATGCCAGTCA
GAACATTAATGTTTGGTTAAACTGGTATCAGCAAAAACCAGGGAAAGCCCCTAAGCTCCTGATCTATAAGGCT
TCCAACTTGCACACAGGGGTCCCATCAAGGTTCAGCGGCAGTGGATCTGGGACAGAATTCACTCTCACAATCA
GCAGCCTGCAGCCTGAAGATTTTGCAACTTATTACTGTCAACAGGGTCAAAGTTATCCTCTGACGTTCGGCGG
AGGGACCAAGGTGGAGATCAAACGAACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGTTAG
>Zt/g4-hzVL4-hIgKCL-amino acid sequence
(SEQ ID NO: 38)
AATMGWSWILLFLLSVTAGVHSDIQVTQSPSFLSASVGDRVTITCHASQNINVWLNWYQQKPGKAPKLLIYKA
SNLHTGVPSRFSGSGSGTEFTLTISSLQPEDFATYYCQQGQSYPLTFGGGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVT
HQGLSSPVTKSFNRGEC*
>G4-hzVL5-hIgKCL
(SEQ ID NO: 39)
GCCGCCACCATGGGCTGGAGCTGGATCCTGCTGTTCCTCCTGAGCGTGACAGCAGGAGTGCACAGCGACATCC
AGATGACCCAGTCTCCATCCTCCCTGTCTGCATCTGTAGGAGACAGAGTCACCATCACTTGCCATGCCAGTCA
GAACATTAATGTTTGGTTAAACTGGTATCAGCAGAAACCAGGGAAAGCCCCTAAGCGCCTGATCTATAAGGCT
TCCAACTTGCACACAGGGGTCCCATCAAGGTTCAGCGGCAGTGGATCTGGGACAGAATTCACTCTCACAATCA
GCAGCCTGCAGCCTGAAGATTTTGCAACTTATTACTGTCAACAGGGTCAAAGTTATCCTCTGACGTTCGGCGG
AGGGACCAAGGTGGAGATCAAACGAACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAGCAGGACAGCAAGGACAGCACCTA
CAGCCTCAGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGTTAG
>Zt/g4-hzVL5-hIgKCL-amino acid sequence
(SEQ ID NO: 40)
AATMGWSWILLFLLSVTAGVHSDIQMTQSPSSLSASVGDRVTITCHASQNINVWLNWYQQKPGKAPKRLIYKA
SNLHTGVPSRFSGSGSGTEFTLTISSLQPEDFATYYCQQGQSYPLTFGGGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVT
HQGLSSPVTKSFNRGEC*

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Ronsin C, Muscatelli F, Maffei M G, Breathnach R. A novel putative receptor protein tyrosine kinase of the met family. Oncogene 1993; 8:1195-202.
2. Wang, M H, Ronsin C, Gesnel M C, Coupey L, Skeel A, Leonard E J, et al. Identification of the ron gene product as the receptor for the human macrophage stimulating protein. Science 1994; 266:117-9.
3. Yao H P, Zhou Y Q, Zhang R, Wang M H. MSP-RON signaling in cancer: pathogenesis and therapeutic potential. Nat Rev Cancer 2013; 13:466-81.
4. Wang M H, Lee W, Luo Y L, Weis M T, Yao H P. Altered expression of the RON receptor tyrosine kinase in various epithelial cancers and its contribution to tumorigenic phenotypes in thyroid cancer cells. J Pathol 2007; 213:402-11.
5. Zhou Y Q, He C, Chen Y Q, Wang D, Wang M H. Altered expression of the RON receptor tyrosine kinase in primary human colorectal adenocarcinomas: generation of different splicing RON variants and their oncogenic potential. Oncogene 2003; 22:186-97.
6. Yao H P, Zhuang C M, Zhou Y Q, Zeng J Y, Zhang R W, Wang M H. Oncogenic variant RON160 expression in breast cancer and its potential as a therapeutic target by small molecule tyrosine kinase inhibitor. Curr Cancer Drug Targets. 2013; 13:686-97.
7. Thomas R M, Toney K, Fenoglio-Preiser C, Revelo-Penafiel M P, Hingorani S R, Tuveson D A, et al. The RON receptor tyrosine kinase mediates oncogenic phenotypes in pancreatic cancer cells and is increasingly expressed during pancreatic cancer progression. Cancer Res 2007; 67:6075-82.
8. Maggiora P, Marchio S, Stella M C, Giai M, Belfiore A, De Bortoli M, et al. Overexpression of the RON gene in human breast carcinoma. Oncogene 1998; 16:2927-33.
9. Lee W Y, Chen H H, Chow N H, Su W C, Lin P W, Guo H R. Prognostic significance of co-expression of RON and MET receptors in node-negative breast cancer patients. Clin Cancer Res 2005; 11:2222-8.
10. Welm A L, Sneddon J B, Taylor C, Nuyten D S, van de Vijver M J, Hasegawa B H, et al. The macrophage-stimulating protein pathway promotes metastasis in a mouse model for breast cancer and predicts poor prognosis in humans. Proc Natl Acad Sci USA 2007; 104:7570-5.
11. Park Y L, Lee G H, Kim K Y, Myung E, Kim J S, Myung D S, et al. Expression of RON in colorectal cancer and its relationships with tumor cell behavior and prognosis. Tumori 2012; 98:652-62.
12. Wang J, Rajput A, Kan J L, Rose R, Liu X Q, Kuropatwinski K, et al. Knockdown of Ron kinase inhibits mutant phosphatidylinositol 3-kinase and reduces metastasis in human colon carcinoma. J Biol Chem 2009; 284:10912-22.
13. Xu X M, Wang D, Shen Q, Chen Y Q, Wang M H. RNA-mediated gene silencing of the RON receptor tyrosine kinase alters oncogenic phenotypes of human colorectal carcinoma cells. Oncogene 2004; 23:8464-74.
14. Qian F, Engst S, Yamaguchi K, Yu P, Won K A, Mock L, et al Inhibition of tumor cell growth, invasion, and metastasis by EXEL-2880 (XL880, GSK1363089), a novel inhibitor of HGF and VEGF receptor tyrosine kinases. Cancer Res 2009; 69:8009-16.
15. Schroeder G M, An Y, Cai Z W, Chen X T, Clark C, Cornelius L A, et al. Discovery of N-(4-(2-amino-3-chloropyridin-4-yloxy)-3-fluorophenyl)-4-ethoxy-1-(4-fluorophenyl)-2-oxo-1,2-dihydropyridine-3-carboxamide (BMS-777607), a selective and orally efficacious inhibitor of the Met kinase superfamily. J Med Chem 2009; 52:1251-4.
16. Pan B S, Chan G K, Chenard M, Chi A, Davis L J, Deshmukh S V, et al. MK-2461, a novel multitargeted kinase inhibitor, preferentially inhibits the activated c-Met receptor. Cancer Res 2010; 70:1524-33.
17. O'Toole J M 1, Rabenau K E, Burns K, Lu D, Mangalampalli V, Balderes P, et al. Therapeutic implications of a human neutralizing antibody to the macrophage-stimulating protein receptor tyrosine kinase (RON), a c-MET family member. Cancer Res 2006; 66:9162-70.
18. Yao H P, Zhou Y Q, Ma Q, Guin S, Padhye S S, Zhang R W, et al. The monoclonal antibody Zt/f2 targeting RON receptor tyrosine kinase as potential therapeutics against tumor growth-mediated by colon cancer cells. Mol Cancer 2011; 10:82-93.
19. Eyob H, Ekiz H A, Derose Y S, Waltz S E, Williams M A, Welm A L. Inhibition of ron kinase blocks conversion of micrometastases to overt metastases by boosting antitumor immunity. Cancer Discov 2013; 3:751-60.
20. Kauder S E, Santell L, Mai E, Wright L Y, Luis E, N'Diaye E N, et al. Functional consequences of the macrophage stimulating protein 689C inflammatory bowel disease risk allele. PLoS One 2013; 8:e83958-67.
21. Guin S, Yao H P, Wang M H. RON receptor tyrosine kinase as a target for delivery of chemodrugs by antibody directed pathway for cancer cell cytotoxicity. Mol Pharm 2010; 7:386-97.
22. Guin S, Ma Q, Padhye S, Zhou Y Q, Yao H P, Wang M H. Targeting acute hypoxic cancer cells by doxorubicin-immunoliposomes directed by monoclonal antibodies specific to RON receptor tyrosine kinase. Cancer Chemother Pharmacol 2011; 67:1073-83
23. Li Z, Yao H, Guin S, Padhye S S, Zhou Y Q, Wang M H. Monoclonal antibody (mAb)-induced down-regulation of RON receptor tyrosine kinase diminishes tumorigenic activities of colon cancer cells. Int J Oncol 2010; 37:473-82.
24. Padhye S S, Guin S, Yao H P, Zhou Y Q, Zhang R, Wang M H. Sustained expression of the RON receptor tyrosine kinase by pancreatic cancer stem cells as a potential targeting moiety for antibody-directed chemotherapeutics. Mol Pharm 2011; 8:2310-9.
25. Sievers E L, Senter P D. Antibody-drug conjugates in cancer therapy. Annu Rev Med 2013; 64:15-29.
26. Ducry L, Stump B. Antibody-drug conjugates: linking cytotoxic payloads to monoclonal antibodies. Bioconjug Chem 2010; 21:5-13.
27. Teicher B A, Chari R V. Antibody conjugate therapeutics: challenges and potential. Clin Cancer Res 2011; 17:6389-97.
28. Yao H P, Luo Y L, Feng L, Cheng L F, Lu Y, Li W, et al. Agonistic monoclonal antibodies potentiate tumorigenic and invasive activities of splicing variant of the RON receptor tyrosine kinase. Cancer Biol Ther 2006; 5:1179-86.
29. Lewis Phillips G D, Li G, Dugger D L, Crocker L M, Parsons K L, Mai E, et al. Targeting HER2-positive breast cancer with trastuzumab-DM1, an antibody-cytotoxic drug conjugate. Cancer Res 2008; 68:9280-90.
30. Brun M P, Gauzy-Lazo L. Protocols for lysine conjugation. Methods Mol Biol 2013; 1045:173-87.
31. Junutula J R, Flagella K M, Graham R A, Parsons K L, Ha E, Raab H, et al. Engineered thio-trastuzumab-DM1 conjugate with an improved therapeutic index to target human epidermal growth factor receptor 2-positive breast cancer. Clin Cancer Res 2010; 16:4769-78.
32. Sharma S, Zeng J Y, Zhuang C M, Zhou Y Q, Yao H P, Hu X, et al. Small-molecule inhibitor BMS-777607 induces breast cancer cell polyploidy with increased resistance to cytotoxic chemotherapy agents. Mol Cancer Ther 2013; 12:725-36.
33. Jumbe N L, Xin Y, Leipold D D, Crocker L, Dugger D, Mai E, et al. Modeling the efficacy of trastuzumab-DM1, an antibody drug conjugate in mice. J Pharmacokinet Pharmacodyn 2010; 37:221-42.
34. Liu C, Tadayoni B M, Bourret L A, Mattocks K M, Derr S M, Widdison W C, et al. Eradication of large colon tumor xenografts by targeted delivery of maytansinoids. Proc Natl Acad Sci USA 1996; 93:8618-23.
35. Lopus M, Oroudjev E, Wilson L, Wilhelm S, Widdison W, Chari R, et al. Maytansine and cellular metabolites of antibody-maytansinoid conjugates strongly suppress microtubule dynamics by binding to microtubules. Mol Cancer Ther 2010; 9:2689-99.
36. Vieira P, Rajewsky K. The half-lives of serum immunoglobulins in adult mice. Eur J Immunol 1988; 18:313-6.
37. Henriksen L, Grandal M V, Knudsen S L, van Deurs B, Grøvdal L M. Internalization mechanisms of the epidermal growth factor receptor after activation with different ligands. PLoS One 2013; 8:e58148-58.

1. An isolated monoclonal antibody that binds human RON, comprising a monoclonal antibody selected from Zt/g4-DM1, Zt/c1-DM1, Zt/64, 3F12, B9, 1G4, or Zt/f2.

2. The antibody of claim 1, wherein the monoclonal antibody comprises: complementarity determining region (CDR) sequences interposed between human and humanized framework sequences; or a human germline framework sequence and CDR sequences interposed between human and humanized framework sequences wherein the framework sequence comprise at least one substitution at amino acid position 27, 30, 48, 67 or 78, wherein the amino acid numbering is based on Kabat.

3. (canceled)

4. (canceled)

5. The antibody of claim 1, wherein the monoclonal antibody is combined with a cytotoxic agent, such that the antibody targets a RON expression protein and the RON-monoclonal antibody and the cytotoxic agent are internalized into the cell, and the monoclonal antibody is bound with a cytotoxic agent, such that the antibody targets a RON expression protein and the RON-monoclonal antibody and the cytotoxic agent are internalized into the cell.

6. (canceled)

7. The antibody of claim 1, wherein an immunoglobulin heavy chain variable region comprises:
a CDR_{H1} comprising an amino acid sequence selected from the group consisting of SEQ ID NOS.: 9 or 15;
a CDR_{H2} comprising the amino acid sequence of SEQ ID NOS.: 10 or 16; and
a CDR_{H3} comprising the amino acid sequence of SEQ ID NOS.: 11 or 17.

8. The antibody of claim 1, wherein an immunoglobulin light chain variable region comprises:
a CDR_{L1} comprising the amino acid sequence of SEQ ID NOS.: 12 or 18;
a CDR_{L2} comprising the amino acid sequence of SEQ ID NOS.: 13 or 19; and
a CDR_{L3} comprising the amino acid sequence of SEQ ID NOS.: 14 or 20.

9. An isolated nucleic acid comprising a nucleotide sequence encoding at least one on an immunoglobulin heavy chain variable region, or an immunoglobulin light chain variable region for a monoclonal antibody selected from Zt/g4-DM1, Zt/c1-DM1, Zt/64, 3F12, B9, 1G4, or Zt/f2.

10. An expression vector comprising a nucleic acid that expresses at least one of a monoclonal antibody selected from Zt/g4-DM1, Zt/c1-DM1, Zt/64, 3F12, B9, 1G4, or Zt/f2.

11. A hybridoma cell selected from a Zt/g4-DM1, a Zt/c1-DM1, a Zt/64, a 3F12, a B9, a 1G4, or a Zt/f2 hybridoma cell that expressed an antibody that binds to human RON.

12. (canceled)

13. (canceled)

14. (canceled)

15. The antibody of claim 1, wherein the immunoglobulin heavy chain variable region that comprises a CDR_{H1}; a CDR_{H2}; and a CDR_{H3} for a monoclonal antibody selected from Zt/g4-DM1, Zt/c1-DM1, Zt/64, 3F12, B9, 1G4, or Zt/f2; and
an immunoglobulin light chain variable region that comprises: a CDR_{L1}; a CDR_{L2}; and a CDR_{L3} for a monoclonal antibody selected from Zt/g4-DM1, Zt/c1-DM1, Zt/64, 3F12, B9, 1G4, or Zt/f2.

16. The antibody of claim 1, wherein the CDR sequences are interposed between human and humanized framework sequences.

17. (canceled)

18. (canceled)

19. A method of treating cancer in a human patient, inhibiting or reducing tumor growth in a mammal, or inhibiting or reducing proliferation of a tumor cell the method comprising administering an effective amount of the antibody of claim 1 to a mammal, tumor or tumor cells in need thereof, or to inhibit or reduce proliferation of the cancer, tumor, or cancer cells.

20. (canceled)

21. (canceled)

22. (canceled)

23. (canceled)

24. An isolated antibody that binds human RON, comprising an immunoglobulin heavy chain variable region and an immunoglobulin light chain variable region having at least a 95% homology to the amino acid sequences selected from the group consisting of:
Heavy chains: SEQ ID NOS.: 2 or 4; and
Light chains: SEQ ID NOS.: 6 or 8.

25. The antibody of claim 24, wherein the immunoglobulin heavy chain variable region comprises:
a CDR_{H1} comprising an amino acid sequence selected from the group consisting of SEQ ID NOS.: 9 or 15;
a CDR_{H2} comprising the amino acid sequence of SEQ ID NOS.: 10 or 16;
a CDR_{H3} comprising the amino acid sequence of SEQ ID NOS.: 11 or 17; and
an immunoglobulin light chain variable region comprises:
a CDR_{L1} comprising the amino acid sequence of SEQ ID NOS.: 12 or 18;
a CDR_{L2} comprising the amino acid sequence of SEQ ID NOS.: 13 or 19; and
a CDR_{L3} comprising the amino acid sequence of SEQ ID NOS.: 14 or 20.

26. (canceled)

27. (canceled)

28. The antibody of claim 24, wherein the amino acid is at least one of SEQ ID NOS: 2, 4, 6, 8, 22, 24, 26, 28, 30, 32, 34, 36, 38 and 40.

29. The antibody of claim 24, wherein the antibody pairs at least one of SEQ ID NOS: 22, 24, 26, 28, 30, with at least one of SEQ ID NOS: 32, 34, 36, 38 and 40.

30. The antibody of claim 24, wherein the antibody is a recombinant antibody encoded by one or more nucleic acids that encode a heavy, a light chain, or both, having at least 95, 98, or 100% identity to at least one of SEQ ID NOS: 1, 3, 5, 7, 21, 23, 25, 27, 29, 21, 33, 35, 37 or 39.

31. The antibody of claim 24, wherein the CDR sequences are interposed between human and humanized framework sequences wherein the framework sequence comprise at least one substitution at amino acid position 27, 30, 48, 67 or 78, where in the amino acid numbering is based on Kabat.

32. (canceled)

33. An expression vector comprising the nucleic acid of claim 9.

34. A host cell comprising the expression vector of claim 10.

35. (canceled)

36. A method of producing an antibody that binds human RON or an antigen binding fragment of the antibody, the method comprising: (a) growing the host cell of claim 34 under conditions so that the host cell expresses a polypeptide comprising the immunoglobulin heavy chain variable region and the immunoglobulin light chain variable region, thereby producing the antibody or the antigen-binding fragment of the antibody; and (b) purifying the antibody or the antigen-binding fragment of the antibody.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. The antibody of claim 1, further comprising a synergistic amount of a chemotherapeutic agent, and
an antimetabolite, a nucleoside analog, or a platinum-based antineoplastic agent, or
at least one of 5-Fluorouracil, Gemcitabine, or Oxaliplatin.

49. (canceled)

50. (canceled)

51. (canceled)

52. (canceled)

53. (canceled)

54. (canceled)

55. (canceled)

56. The isolated nucleic acid of claim 9, wherein the nucleic acid has at least 95%, 98%, or 100% sequence identity with at least one of SEQ ID NO: 1, 3, 5, 7, 21, 23, 25, 27, 29, 21, 33, 35, 37 or 39.

57. (canceled)

58. (canceled)
